SEQUENCE LISTING (9 sequences)

SEQ ID NO: 1 - DNA, 8366 bp, Capra hircus
accaaagtgattatggctgagggattgttcccacaccaacgcttgaaaaatatccaatttcaaacacagtaagctgcatcaaaaaaaaattatccatgaaaactgctttatcttactttaatattgaaaatgcatttcaaggtaaccagaaaaaagacccacttctacccactaatatgatttacagtaaaatctgctcttaaaaaaattactttagaacttagtcttaaatgtaattaatgttttgctgttatgcagtcgccaccgaaattctctctaaattcataagaaacattcaatatgattaagtccaaccaaaaaaaggaataacatttaaaatatcaagatgatataccttaaaattttatatatgggaaaaatctaagaaaaatagttacaatctacgtaacgtatcttgaaatttatcaaatataacatataaaaaggcgtcctttgaaataattacggcaaatagctcctaaatagtaagacacttaagctacacgctttaatttccgaatgagcagaacgaaaatttaaactaggcaccgaaagttattgtcaggatataatcttacttataggatataatctttaataagactagggaaaaaaaaaaaaactttcctaagagcacaccaaactgttttttatgtacacaaatgtcatgcagctttaacgaacgaatgcctgcgttccctggtaagaaaatgttacagggcgtatatcatttaaaaaatacagaatgcaaatttaatccacatctaaaagctaaagctcaataaggtttaaaaagctcttacattacttaaaacacctgtaggagaatgtgcacagtggcatattcaagtaacattttagcagaatttcgtaagtatgtgctacagaactgtaatatttcataataacgaaaaattttcaatatttgaaacgtaatggatcacataacaaaaatgaaattgtactacttaagaaactgagtgaagctactacttctgtagcaaagaaagaaaaaggtaaaagcaaatgtggaacagattatcttcaccaccacaccttccattaaccctcagccataagcggccgcgaagttcctattctctagaaagtataggaacttctacttgaatgcagttatagaaagtaccttatctattcatcattgctatggaatacataagagtcttctatattcaaggtattttatattcaattttgacgtaaaaacaaaaaggaaaagattttaatgtttgtgttcttttgcattgctgttagtgtggatggactcggtgtccatgagcaagtataaataaaccagccggtgcaaatcttgttcttttggggcaagccttttgcattttaaaagcaacatcacatgctttgcatacctacctttccacatctttatactatcataacatggcttgggcagatgaaatccagaaagttttgactatgcttatgtattgacagtctaatattttatgtgaaaaagtaaacaaagcaggactatgtattgaatttggcagcctatttccttgttctatgaatatgcaaaaaatacagctgttcagagtttgtccacgatcccacaagaacagcactaacatactgatcggctgactgtattctaatatttccctgttaggcgtgtccacaaatcccaaaatgtgcactgagagatttgcctgctgccacaggcagccaactctgcttaagaggaaaacatcacttgactatctaagtgctttatgcttcatataaacaaacaagcaaaaaaattgtataacagaatctgtctccagacttgcagattgtcgagaaggacatgatgtctgttggggtagtcgaaaggcatggaacatatttaggtatatttactcttcttacctttcaggaaaaaccaaatgacctactgttataaagaatattgctttttattccctcagtaccaaatcaataaaagtcctttcagcacaacagctgttccttaatggccccagttaatgcactgcctgaatcaaacacattccagtgtctacaccaggcagaccctcaattaatggtgatggatcgactttctgacaagcagtagaaactcaatttatagaaattgatcctatgagacaaaaataaataatttaagagcactgtgtgttaaattctatgtttggcaacataacaatgtgtatagatctaagaactatgtgtattttttttaatgaaaaggttttccagttagctgacagatactgagtgtctcaatcattttgagttataatccaagcatagcaaaactataaatctgggtgattcatatgaatattctgatagtgatatcataatgattatgaagaatctgaagactcgtttacctaaagataagacacatgtttgttcgttagtaattataactttaaatcttgcaataaagattgcctattagtgataaacactatttgaattgtccatttcagatttttagtgtacagattttcataactttctacattattgtacactattctttaaggaagatgagcaggtttgaaagagggccagaagtaaccacttaatttgaaagtataatgaattttctctttcgctgccatagaactgtgtggttttaccctttgccaggtgacagcatctccttagtcaaagcagagtcttagaaaatgagggaatgggctgttgggtacttcagcagagcattgctcttatatcctatgtccaaaccagagcactagctgcgctctacattccaaaaagtttgagcaaataaactgacaagcatctaccactacaagatgcaaccagacaggagcttccagagttctgcctctaggtacaggcaaagttctttcacatatgatgtgaagcgagacgtgtacaatgaggaaaactttcaacaggaacacagaaagaagactgcttcctccgggaacgtggacatcgacatcagcaccgtcagtcaccacgtggagtgcagatgctcatggtacaagttccgaagatgcctgcttaccgtgtttcccttcctagagtggatgtgtttctatcgattcaaggattggcttcttggagacttacttgctggtataagtgttggccttgtgcaaattccccaagtcctgatgcttggtttgctggcaaggcatctgattcctcctctcaatgtctcttatgcagctttctgtgcttcagtaatttatggaatttttggatcatgtcatcaaatgtccattggtacattcttccttgtgagtgctctgacaatcaatgtcctgaggacagagccattcaacagtggccacttattactgggaactttcatccaggatgacttttctaacataaccttctatgagaactataacagatccttgagttcggtggcatctgtaactttgctaactgggattattcagctgtctatgggcatgttaggttttggcttcattgtcacttacattccggaggctgcaatcagtggttacctggctgccacagccctgcacattatgctgtcccagttgacctgcatcttcggagttatgatcagttacaattctggtcccatcgccttcttctacaacataattaactactgtttaggtctccctaaagctaattccaccagcatcttactatttctaaccactattgttgctctgagaatcaacaaatgtatcagaatttccttcaatcagtatcccattgaatttcccatggaaatttttctgatccttggctttgctgcattttcaaacaaggtaaacatggccacggaaaacagcctgatgctcattgagatgataccttacagcttcctgtttcctgtaacgccagatatgagcaatcttactgaagttcttgcagaatcgttctccttagctttagtgagctcgtttttgctcatatttctgggcaagaagattgccagtttccataactatgacgtcaattccaaccaggatttaatagccattggcctttgcaatgtcgtcagttcatttttcagatcttatgtgtttactggtgctgttgccaggaccattattcaggataaaactggaggaagacaacagtttgcatctctggtaggcgcaggcctcatgctgctcctgatgatgaagatggcacactttttctacaaactgccaaacgctatagtggctggtattatcctgagtaacgtcctaccctaccttgaagttgtttacaccctacccagtctgtggaggcagaaccagtatgactgtctcatttggatggtgacgttcatgtctgcaattttactgggactggatattggactagtcgttgcagtaacttttgccttcttcatcatcactgttcagtcacacagaactaagattctcctcctgggtcagatccctaacaccaatatttatagaagcttccaagactatcgggaggttgcaaacattccaggggtgaagatcttccagtgctgcaacgccatcacatttgtcaatgtccactacctcaagcgcaaggtgttagaggagattgaaatggtaaagatgcctcttacagacgaggaaatttataccctgttcagtccaaatgaagagggcgcacagcgaggaaagatttgccggtgttactgcaactgtgatgaaccagagccatcgcccagggttatttacacagaacgatatgaagttcaacggggccgagagtcctccttcattaacctggtccgctgctcacgttttgagagcgtggacacaggccaaagtatgtctgaagaccaagtaccgtacataacatcttcctcgtctcagagaaacccaaactatgaggaggtggagaaagtctggctttctgatgacccctccaggagcatgacgatcacactccctgaggcttctaatactcaggtcagggctacaaaactcctgccttactcaacttcaactgttctacccagcgtccacaccatcatcttggacttctccatggtacatcttgtggacgcacaggctttggtcgtattaaggcagatgttctgtgctttccaaaacgtcaacatcttggtgctcattgcagggtgtcactcttttgtggtcaggtcacttgagaagaatgatttctttgacgctggcatcactaaggcccagctgttcctcaccctccacgacgctgtgctgtttgctttgtcaaggaagctgccagagtcctcggagttaagtgtggacgaatcaaagaccgtcatacaggaaaccttctcagagacagacaagaaagaagaatcaagacataaaacaagcagaagttttatagaagcccccagaagtaaaagtccagccttctccttactcccagacccagagatggaggaggaatcagacttggatctgtattccacgatacagatgtctaaagaccatgggctggatctggacctagacctggatcgtgaggtggagcctgagtcagagctggagcctgaatctgagctggatcaagagacagagctcgagcctgacccagaggccagtcacaagccaactaggcagaagtactggtctctgtttagggctataattcccagatccccaactcacactcaggctaggacacagtcggtagacaggaggcatcaaaatgtgaaaccatatacatccaaggctgacaccagtgaggagatcgattataaagatcatgatggcgattataaagatcatgatattgattataaagatgatgatgataaatagattttctctacccacagcgtttgatgtattattattattatttttgcaggcctcagtaatttgggattatgaatgggattctattttaccaaagtaattcaatttttataatcaagattctatttttgagtttcaaagagaaattatatattcttctaccaaagattgattacaagcaaggctacttagggattagttttggtttaaagagaatgaagactgaataaaataaaatcactagaaaattgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagttcctattctctagaaagtataggaacttcggtacctcactttggtgtagtattcgggaagtgcacaaacgtcaaaatctgacagacatgcggagctggagcccaactccacttcaaatcgctcagtctcagtgacggcgtttgccgttaagtgcaacgaggagaccaatctcctctgaggattgtgccacaattcccgaaggtggggcggttagcctcccggcacaggaaaaaaaaaaaaagcttaccggaggaaaaaaaaggcgggggggggacggggggtggagacagaaacaagagccattttcctttacagttcaaaacacaggcacttttatagagcgccggatccgcacccattagccgggttagagacactccacccgggtggtggtggacaaaagagggcctaacagcccccaccccaacaaaaggacgaagatatacttaacaagctccagccgctgaatccctgaggaggtgcggaggggtggctcctcactgtttaatgtagggccttttacctcaaaaacaaactacgcgacagcgaaaggaaatacaaagctcaacccccagtaccggaaaaaaaactgccacggccgagctccggcctgtagcctgagggaaaaaggccctgctcccaactccacacgttgcggccctcttacctcttttcttccgcgcggtggcgtttctctgtcgttagatgggctccaggtggaccgtttctcctgtttaacgtccgcttagtgctcccgcagctgcggtttcaccttttagggcctggtctcttcaccccgaatctgtcctagctgccaacaccaatactaaaatgagccgctaacgcggtagtctcccgtaaaggagaaggcaacacaagcagtcaagctcctgaaaggcctgggagctgagtgacgcttgaaggggctgggctccacaaaaccccttcagggcgggatacgaggccgctggagttccagaaatcgcttgccccggccctcttcaatatggcggattcagcaagccaaaaaaaaacggccgaaccaccggcaaccgcgacggcgcaaactcccaccagcctcagcgccagtcggagcctccaatatgacgccgcgccacgccgctccgccccctcgaagtcccccctctcgactccgcctccttatcctgcctccctcagccataatcactttggt

SEQ ID NO: 2 - DNA, 1694 bp, Mus musculus
caaaaccacgtatatccctgtacaaaagcaaggggtgtatttatagggggcaaatgacatactatttcctgtcaatcctttgctggaatggaacatgcaggaatgtacatgtacatgtgtgcaattcagcctatgacacatgtcaaagatgatttagtgtactaagtctttcattctgaggtggaagcctgtctcctgggctgatcataagtaatgtagcaagacagatctccttcctttgggactagctctttgcatagtaaaagcaatggcaaatgagtggcacaaagctttgcatatgtacctctctgagtctttactcaggatgctttgactatgtttagtctttatcttcagctcaacatatttaatgtgcaaaagtaaacaaagcaggaatgagtacttagtctggtggcccatttccttgttgtatgaatattcaaaattccacctgctcaaacttcacacatgatctctgcaaaacagcagtgacagcttgattagatagttttattcaagtattttataattaaatgtactaagacacactacagtgtaagacacacttgctgttacaggcagatagctgtgtttataagaagaatattgtttgacagtctactaaataccttttgcttcagataaacaaacaaaaaaatgtgtaaaaatcttctcttcccttgaggcagccaattttcaaaaatttttaggatgttagtagggtaggtaaaaaattatggaacatagttgggtatatttagtattttgattatcatctgattatctttgatatttttatatgcttccaaaacattggtccttattccgtaagtatcaactcagactgtgtacccccaacacaacagctgttgcttaatggccctaaaatgttaagtgctgtccacattgaacacagatcagtgtccatacaaagtaggccttcaattaccagtgatggattgactttcacacaggcagtaaaagctcaattcatagtgattaatcctatgagaataaaaatccatgttaagatactggacctagaattttacatttatagctaaaaaaaaaatgtataaatatgtaaatcatgtcctttgttgctattatttaaatgttttctaatttattgatatattaagagattctcaatatttgtaccaaccttagaataggcaaaatggaatttgaatagcatcatgcataatctttttgaggctaaggggtaattatgaagaatctgaaaaacaaagtatttaaagttaaaaatgcaagtgtttttgattaatagtaaatacaataccaataataaatattttctgttggtaagaaacatttcacttgaattgcccatcacagattttctttaatttcaagattttgaatgctttccatgtgttttgtgctaaactctcaggcaggtaagcatgattttgaaggcgggccagatgtgaccacttaatttaaaagcataatgagtttcctcttaagcttccattggagttgcatggtttttcctcttgatcaggtgacagatggactagatccccttagtcaaagcagaattgagagcgcaggggtagctgctgcctgttgtagcgagcaccgctcatatgtcctatatctaaactacagctgtgctccgtgtttgaaaagtttgagcaaatcaactgcccagccactaacatcaaaag

SEQ ID NO: 3 - DNA, 1634 bp, Homo sapiens
caaatctccttcctttgggacaggccctttgcattgtaaaagcaataccacatgatttgcacaaatccttgcctatccaccactgcacatctttatactatcataatatggctttggaagaacggaacccaggaagttttgactatgtttatgtatcttcagcctaatatttttgatgtggaaaaagtaaacaaagcaggactatgtatttaatctggcagcctattttcttgttgcatgaatatgtaaaaactcacctgttcaaactttgcacatgatcccacaagaagagcaccactgacaggttggtggattgactgtattctggcattttctggttaagcacattcacaaatcccaaaatgcacactgaaagatgtgccacaggcaaccgactctgttcataaggaaaatattgattgactatctaccaagcactttatgcttcatataaataaaaaattgtgcaacataatctctgccctcaatcctgaagattttcaagaagtacatggtatctgttggggtagttgaaagctatggaacttatttaggtgtaattactgtttttagtttgtaggaaaaaccaaattacctgctgtttcaaagaacattggtttttattcccttggtaccaaatcagtaagagtccctccagcacagcagctgttgcttaatggccctaaagagttaatgcgctgcctaaattgaacatagttcagtgtctacaccaagtgggccctcaattagtgttgatggattgactttctcacaggcggcaaaagctcaatttatagagatggatcctatgacactaaaatcaataatttaagaaaactgtgttgaattttatatttgtagcaatacaatatgtacaaatataaaaactatatgtatttgtatccattgttaaaaaaaagattttttttgttccctgacagatgctaatagatgctcaatagctttaagtaataatctaagcataggtagaccataaaagttggataacttacatgtatatattctgattgtgatataggagtgattataaagaaattgaagggccaaatatctgaagataagatgtaagcatcgctgattagtaataataacttttaatcacagtaataaaaattgcccagtagtgagaaacattctatttgaattgcttattgtagatcatctctagtgtatagattttgacaactttctacattattttatactataaagcagataagcctgttttcgaagatgtgcccgaagcagtcatttaatttgaaagcataatgaacttcctctctagcctccatcaggggggtatggttttaccacctgatcaggtgacagatgagaaagcatctccttagtcaaagcagaatcttagagcattagggaatggactgctaggtacttcagcagagcattactcttgcctcctatgtccaaaccacagctgtgctcaacatttgaaaaatctgaacatataaactgacaagtatctaccaccacaagatgggaagtggaattagttcagagagcaaggagtcagccaaaagatcaaaagaactggagaaaaagcttcaggaggatgctgagcgagatgcaagaaccgtaaagctgctactattagg

SEQ ID NO: 4 - DNA, 1702 bp, Bos taurus
attttaatgtttgtgttcttttgcattgctgttagtgtggatggactctgtgttcatgagtgagtataaataaaccagctggtgcaaatcttgttcctttggggcaggccctttgcattttaaaagcaacatcacatgctttgcatacctacctttccacatctttatactatcataacatggcttgagcagatgaaatccagaaagttttgactatgcttatgtattgacagtctaatattctatgtgaaaaaagtaaacacagcaggactatgtattgaatttggcagcctatttccttgttccatgaatatgcaaaaatacacctgttcagagtttgtccacgatcccacaagaacagcactaacatgctgatggactgactgtattctaatatttccctgttaggcacgtccacaaatcccaaaatgtgtactgagagatttgcctgctgccacaggtagccaactctgcttaaaaggaaaacatcacttgactatctaagtgctttatgcttcatataaacaaacaagcgaaaaaattgtataacaaaatctctgtccccaaacttgcagattgtcgagaaggacatgatgtctgttggggtagtcgaaaggcatggaacatatttaggtatatttactattcttaccttgcaggaaaaaccaaatgacctactgttacaaagaatattgctttttattccctcagtaccaaatcaataaaagtccttccagcacaacagctgttccttaatggccccaaagagttaatgccctgcctgaatcaaacacattccagtgtctccaccaagcagaccctcaattaatggtgatgaattaactttctcacaagtagtaaaagctcaatttatagaaattgatcctatgagacaaaaataaataatttaagagcactctgtgttgaattatatgtttggcagcatagcaatgtgtatagatttaagaactatgtatatatatattttttaatgaaaaggttttctagttatctgacagatactaagagtgtctcaatcattttgagtcataatctaagcatagcagaactataaatctgggtgattcaatataaatattctgatagtgatatcacagtgattatgaagaatctgaagactcacgtacctaaagataagacacacgtttgttcgttaataattataactttaaatcttgcaataaagattgcctattagtgataaacactatttgaattgcccatttcagatctttagtgtacagattttcataactttctacattattgtatgctattctttaaggaagatgaacaggtttgaaggagtgccagaagtaaccacttaatttgaaagtataatgaattttctctttagctgccatagaactgtgtggttttaccctttgtcaggtgacagcatctctttagtcaaagcagagtcttcgaaaatgagggaatgggctgttgggtacttcagcagagcattgctcttacatcctatgtccaaaccagagcaccagctgcgctctacattccaaaaagtttgagcaaataaactgacaagcatctaacactacaagatgggaattggaattagttcagagagcaaggagtcagccaaaagatcaaaagaattggagaaaaagcttcaggaagatgctgagcgagatgcaagaactgtcaagttgctgttgttagg

SEQ ID NO: 5 - protein, 60 aa, Mus musculus
EGITKAQLFLSLHDAVLFALSRKFSEPSDLSMDETETVIGETYSESDKNGNLSNLRLKTG

SEQ ID NO: 6 - protein, 57 aa, Homo sapiens
AGITKTQLFLSVHDAVLFALSRKVIGSSELSIDESETVIRETYSETDKNDNSRYKMS

SEQ ID NO: 7 - protein, 57 aa, Sus scrofa
SGISKAQLFLTLHDAVLFALSRKLPDSSELSVDESETVIQETYSETEKNGESRHKMK

SEQ ID NO: 8 - protein, 57 aa, Capra hircus
AGITKAQLFLTLHDAVLFALSRKLPESSELSVDESETVIQETFSETDKKEESRHKTS

SEQ ID NO: 9 - protein, 57 aa, Bos taurus
AGITKAQLFLTLHDAVLFALSRKLPESSELSVDESETVIQETFSETDKKEESRHKTN

(19) United States
(12) Patent Application Publication
(10) Pub. No.: US 2020/0399661 A1
(43) Pub. Date: Dec. 24, 2020

(54) MATERIALS AND METHODS FOR PREVENTING TRANSMISSION OF A PARTICULAR CHROMOSOME

(71) Applicant: Aggenetics, Inc., San Diego, CA (US)

(72) Inventor: James WEST, Nashville, TN (US)

(73) Assignee: Aggenetics, Inc., San Diego, CA (US)

(21) Appl. No.: 16/975,959

(22) PCT Filed: Feb. 26, 2019
(86) PCT No.: PCT/US2019/019655

§ 371 Date: Aug. 26, 2020

Related U.S. Application Data
(60) Provisional application No. 62/635,270, filed on Feb. 26, 2018.

(51) Int. Cl. C12N 15/90; C12N 5/076; C12N 15/113; A01K 67/027

(52) U.S. Cl. CPC C12N 15/907; C12N 5/061; C12N 15/113; A01K 67/0276; A01K 2227/105; C12N 2830/007; C12N 2310/531; A01K 2217/058; A01K 2217/075; C12N 2310/141

(57) ABSTRACT

Provided herein are material and methods for changing gene expression in select sex chromosomes. The materials and methods of the subject invention can be used to produce non-human transgenic animals that produce progeny of a predetermined gender and to generate non-human transgenic animals that produce single-sexed semen.

### CROSS REFERENCE TO RELATED APPLICATIONS

This application claims the benefit of U.S. Prov. App. No. 62/635,270, filed Feb. 26, 2018, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 186122000940SEQLIST.txt, date recorded: Feb. 26, 2019, size: 20 KB).

### FIELD OF THE INVENTION

This invention relates to the field of sex specification in livestock, prevention of transmission of the Y chromosome, and other uses related to transmission ratio distortion (TRD) and preventing (or ensuring) transmission of other chromosomes.

### BACKGROUND OF INVENTION

Sex determination is an important issue in livestock. The ability to create offspring of a specified gender, usually female, has high commercial value in many livestock industries. For example, the dairy cattle industry has a preference for female offspring. Other types of livestock operations also have a preference in order to avoid problems associated with neutering or in order to ensure larger male offspring, e.g., in the meat industry.

Isolated high purity X chromosome bearing or Y chromosome bearing populations of sperm cells can be utilized to accomplish in vitro or in vivo artificial insemination or fertilization of ova or oocytes of numerous mammals such as bovids, equids, ovids, goats, swine, dogs, cats, camels, elephants, oxen, buffalo, or the like.

A number of techniques have been devised to separate sperm, directly or indirectly, based on differences in size, mass, or density of X chromosome and Y chromosome bearing sperm cells. However, almost all of these methods are based on mechanical sorting of semen and have the potential to damage sperm cells, thereby reducing pregnancy rates and increasing costs. Furthermore, staining of spermatozoal DNA can have detrimental effects on fertilization rates, due, e.g., to the amount of DNA stain present in sperm cells, the time elapsed due to staining procedures, and a reduced progression of fertilized oocytes to blastulation. Additionally, the purity of sperm cell selection can be negatively impacted by overlapping ranges of fluorochrome DNA staining patterns in the X and Y chromosome-bearing sperm cell populations.

Genes that control sex determination, i.e., start the ‘male’ or ‘female’ cascade, are known and identified. However, methods using these genes have produced suboptimal results because, e.g., the resulting animals lost many sex related characteristics and appeared to be more androgynous or of mixed phenotype than truly of the forced sex.

Other methods used site-specific nucleases such as CRISPR to attack the Y chromosome and prevent its transmission. These methods had practical problems because of their modest overall efficiency due to either no cleavage in some instances or cleavage repair by non-homologous end-joining in others, and have potential regulatory problems.

### Spermatogenesis and Cytoplasmic Bridges

One aspect creating difficulties in producing single sexed animals is that fact that during spermatogenesis cytokinesis is incomplete and germ cells that arise from the same undifferentiated spermatogonium remain connected to each other by intercellular bridges that persist until late spermiogenesis.

Spermatogenesis in all mammalian species takes place primarily in seminiferous tubules in the testes. It takes place roughly “outside in”, in which the earliest stages of development are near the margin of the tubule, and the latest stages are near the center, with the mature sperm released into the center of the tubule. The initial cell in spermatogenesis is the spermatogonial stem cell. These stem cells are capable of self-renewal and differentiate into spermatogonia. There are several rounds of mitotic proliferation, followed by Meiosis I, in which the chromosome pairs separate, and then by Meiosis II, in which the chromatids separate into haploid spermatids. Importantly, throughout this process, the sperm are all connected by cytoplasmic bridges, through which both RNA and proteins diffuse freely. Thus, spermatids share transcripts and/or gene products across the cytoplasmic bridges.

In the final stage of spermatogenesis, spermatid elongation, the cytoplasmic bridges break; although they persist in residual bodies. By this point, transcription is shutting down as the chromatin is being condensed into sperm heads. Therefore, sperm are primarily functionally diploid throughout their generation, even though after Meiosis II they have haploid genomes.

The shared cytoplasm throughout most of the development cycle is an obstacle in that proteins produced in one spermatid can leak over to another spermatid. The cells are, thus, functionally diploid and cytoplasmic granules loaded with RNA and RNA binding proteins move between the spermatids in a microtubule-dependent manner.

### Transmission Ratio Distortion (TRD)

The exchange of transcripts and gene products across cytoplasmic bridges during spermatogenesis would suggest that any transcript or gene product of a gene inserted on the Y chromosome will just spread to sperm carrying the X chromosome through the cytoplasmic bridges. However, distortion of inheritance from the natural Mendelian ratio, referred to as Transmission Ratio Distortion (TRD), exists.

The best-studied example of TRD is the t-complex responder (TCR) system. In this system a naturally occurring mutation on mouse Chromosome 17 does not affect chromosome transmission in eggs but leads a heterozygous male to pass this mutation on to nearly 100% of his offspring. Although the mechanisms involved in t-complex are complicated, a key finding provides that attaching the untranslated regions (UTR) of the TCR to a construct prevents such construct from being shared between sperm via cytoplasmic bridges. The 5′ and 3′ UTR of the TCR system contain sequences that bind them to a cytoskeleton structure, preventing them from being moved through the cytoplasmic bridges. The fact that there is nearly perfect TRD with TCR demonstrates that the restriction does not just lie in the RNA, but extends to the protein, likely because of membrane insertion sequences in the protein itself.

A tethering through the UTR coupled with membrane insertion is also seen in Sperm Adhesion Molecule 1 (Spam1), a protein responsible for penetrating the egg, which also does not cross the cytoplasmic bridges because of tethering to cytoskeletal elements.

There are numerous other examples of TRD in nature, and TRD is not restricted to mice. Strong statistical evidence indicates numerous sites of TRD in cattle, where TRD more commonly occurs in males than females.

Another example of TRD that crosses species is the Slx/Sly conflict. This system comprises a set of homologous genes on the sex chromosomes that are in competition with each other. Slx promotes a skew towards more female offspring; Sly promotes a skew towards more males¹. Although the strength of these skewing genes might not be strong enough for the purpose of the subject application, the Slx/Sly conflict demonstrates that sex chromosomes are not immune to this phenomenon.

### Gnat3 and Tas1r3 Chemoreceptors

Another striking example of TRD is found in chemoreceptors gustducin alpha-3 chain (Gnat3) and taste receptor type 1 member 3 (Tas1r3). These transmembrane proteins are involved in the ability to sense the flavor “umami”—best characterized by monosodium glutamate. Sperm lacking both Gnat3 and Tas1r3 never produce offspring but transmission on the female side is unaffected. Studies indicate that the failure to produce offspring is due to a loss of progressivity in sperm, i.e., sperm cells are correctly formed and can move as well as other sperm but cannot swim along a chemical gradient, which means the sperm cannot find the egg.

Importantly, unlike the t-complex that is only found in mice or Spam1, for which the required additional hyaluronidases appear species specific, the Gnat3/Tas1r3 system is extraordinarily well conserved across species. The 5′ UTR shows very high sequence homology between mice, humans, pigs, and cattle. A multiple sequence alignment across 90 species showed that the Gnat3/Tas1r3 system is highly conserved in all placental mammals. In contrast, the UTRs in other taste receptors have almost no sequence conservation across species.

In keeping with the previous examples of TRD, both Tas1r3 and Gnat3 are membrane inserted proteins comprising membrane-insertion domains and are expressed very late in spermiogenesis, i.e., only in spermatids.

In cattle, for example, Gnat3 does not appear to be active in sperm until capacitation, when it becomes localized to the sperm axoneme near the mitochondrial bundles, presumably sensing chemical signals and guiding activation of the sperm tail.

### BRIEF SUMMARY

The subject invention provides materials and methods for preventing and/or inhibiting transmission of a particular chromosome or, alternately, forcing transmission of a particular chromosome.

In specific embodiments, the methods provided comprise inserting sequences into a particular chromosome, for example, the Y chromosome, which inserted sequences prevent Y chromosome sperm from successfully fertilizing an egg. The methods provided make use of, for example, transmission ratio distortion mechanisms.

In other embodiments, sequences are inserted that require transmission of that chromosome by using a distorter-responder system.

In specific embodiments, methods are provided for the production of single-sexed X chromosome or Y chromosome semen.

Further provided are genetically modified animals that produce offspring or progeny of a single sex.

In some embodiments, methods are provided for immobilizing sperm that produce a particular sex. In other embodiments, methods are provided for deleting sperm that produce a particular sex.

In preferred embodiments, materials and methods are provided for the production of single-sexed semen comprising at least 90% X chromosome sperm cells, which semen can be used to generate female animals.

Female animals can also be carriers of a transgene that is introduced on at least one sex chromosome and such female carriers can be bred naturally to propagate the desired trait. In such breeding methods, progeny may be generated using natural breeding techniques, there by having one copy of said transgene. Alternatively, progeny may be generated from intracytoplasmic sperm transfer from a carrier male that produces substantially male progeny, thereby having two copies of said transgene.

In further embodiments, transgenic female animal are provided for producing male animal that produce substantially female progeny.

In further preferred embodiments, materials and methods are provided for the production of single-sexed semen comprising at least 90% Y chromosome sperm cells, which semen can be used to generate male animals.

Advantageously, in some aspects, the animals generated using the materials and methods of the subject invention are not genetically modified.

Also encompassed are the genetic constructs and tools used to accomplish the methods described herein.

### BRIEF DESCRIPTION OF DRAWINGS

FIG. 1A shows a construct comprising sequentially arranged promoter/shRNA units comprising two U6 promoter/Tas1R3 shRNA units and two U6 promoter/Gnat3 shRNA units. FIG. 1B shows a construct comprising divergently oriented promoter/shRNA units comprising U6 and H1 promoter/Tas1R3 shRNA units and U6 and H1 promoter/Gnat3 shRNA units.

FIG. 2A shows a construct as shown in FIG. 1A and additionally comprising a Gnat3 promoter operably linked to a “wobbled” Gnat3 gene. FIG. 2B shows a construct as shown in FIG. 1B and additionally comprising a Gnat3 promoter operably linked to “wobbled” Gnat 3 gene.

FIG. 3 shows a cross section of a seminiferous tubule of a wild-type animal labeled with an anti-body to Slc26a8 protein, which is a membrane-inserted protein that is only expressed in late spermatids (reproduced from (1)).

FIG. 4A shows a construct comprising a Gnat3 promoter operably linked to a Gnat3 5′UTR and a Slc26a8 dominant negative gene. FIG. 4B shows a similar construct as in FIG. 3A but with a GFP gene flanked by loxP sites for removal via application of Cre recombinase fused 3′ to the Slc26a8 dominant negative gene and homologous arms 5′ and 3′ of the construct. FIG. 4C shows Gnat3 5′UTR tethering RNA transcripts to the cytoskeletal structure of sperm cell. FIGS. 4D-E shows Slc26a8-flga staining in testes (FIG. 4D, where red indicates flag and Slc26a8 dominant negative mutant, and blue indicates nuclei) and sperm cells (FIG. 4E, where green/yellow indicates flag and Slc26a8 dominant negative mutant, blue indicates nuclei, and red indicates the mitotracker that stains the sperm mid-piece). FIG. 4F shows decreased sperm motility in SLC26a8 dominant negative transgenic mice as compared to wild-type.

FIG. 5 shows a construct comprising a TCR/Smok2b promoter sequence, a TCR/Smok2b 5′ UTR, a Slc26a8 dominant negative gene, a TCR/Smok2b 3′UTR with a Smok2b intron sequence, and a poly A.

FIG. 6A shows the construct used to prevent and/or inhibit RNA transfer in sperm cells by tethering a RNA to a cytoskeleton structure (reproduced from (3)). FIG. 6B shows a cross section of a seminiferous tubule of a wild-type animal labeled with a Smok-specific probe (reproduced from (3)). FIG. 6C shows a cross section of a seminiferous tubule of a non-human transgenic animal expressing the RNA tethering construct labeled with a myc-specific probe (reproduced from (3)). FIG. 6D shows a cross section of a seminiferous tubule of a wild-type animal labeled with a myc-specific probe (reproduced from (3)). FIG. 6E shows a schematic of a cross section of a seminiferous tubule (reproduced from (3)). FIG. 6F shows fluorescence microscopy images of cross sections of seminiferous tubules of transgenic animals labeled with anti-myc and anti-tubulin antibodies and of wild-type animals labeled with anti-myc antibodies (reproduced from (3)). FIG. 6G shows longitudinal sections of seminiferous tubules of wild-type and transgenic animals labeled with anti-my antibodies (reproduced from (3)).

FIG. 7A shows the construct used for the translation delay strategy to prevent protein expression until after cytoplasmic bridges between sperm cells are broken (reproduced from (3)). FIG. 7B shows the cross section of a seminiferous tubule of a wild-type animal and a non-human transgenic animal expressing the construct of FIG. 6A labeled with anti-myc antibodies (reproduced from (3)). FIG. 7C shows fluorescence microscopy images of a cross section of a seminiferous tubule of a wild-type animal and a non-human transgenic animal expressing the RNA tethering construct labeled with fluorescent anti-myc antibodies (reproduced from (3)). FIG. 7D shows a schematic of two chromosomes expressing different alleles that affect sperm functionality resulting in differential transmission of the respective chromosomes and non-Mendelian inheritance (reproduced from (3)).

FIG. 8A shows a genetic construct comprising elements of the goat Gnat3 promoter and 5′UTR in combination with a goat SLC26a8 dominant negative gene used for preventing and/or inhibiting transmission of an arbitrary chromosome in goat. FIG. 8B shows the E to K mutation in the goat Slc26a8 gene making it dominant negative. Sections of amino acid sequences for mouse SLC26a8 (SEQ ID NO: 5), human SLC26a8 (SEQ ID NO: 6), pig SLC26a8 (SEQ ID NO: 7), goat SLC26a8 (SEQ ID NO: 8), and cattle SLC26a8 (SEQ ID NO: 9) are shown.

FIG. 9A shows a pair-wise sequence alignment of Gnat3 promoter and 5′UTR sequences between cattle (nucleotides 201-1523 of SEQ ID NO: 4) and mouse (nucleotides 322-1635 of SEQ ID NO: 2). FIG. 9B shows a pair-wise sequence alignment of Gnat3 promoter and 5′UTR sequences between cattle (nucleotides 201-1702 of SEQ ID NO: 4) and human (nucleotides 130-1634 of SEQ ID NO: 3). FIG. 9C shows a pair-wise sequence alignment of Gnat3 promoter and 5′UTR sequences between goat (nucleotides 1441-2803 of SEQ ID NO: 1) and mice (nucleotides 322-1687 of SEQ ID NO: 2).

### BRIEF DESCRIPTION OF SEQUENCES

SEQ ID NO: 1 shows the nucleotide sequence of a genetic construct comprising elements of the goat Gnat3 promoter and 5′UTR in combination with a goat SLC26a8 dominant negative gene for preventing and/or inhibiting transmission of an arbitrary chromosome in goat. Sequentially, the elements are: nucleotides 1-23 CRISPR site, 24-1079 left arm (match to goat Y chromosome), 1080-1087 NotI site, 1088-1121 FRT site, 1122-2811 goat Gnat3 promoter and 5′UTR (tethering region), 2812-5750 goat Slc26a8 with E to K mutation making dominant negative, 5751-5996 Spam1 3′UTR, 5997-7166 rabbit beta globin PolyA sequence (including last intron), 7167-7200 FRT site, 7201-7206 restriction site, 7207-8343 right arm (match to goat Y chromosome), and 8344-8366 CRISPR site.

SEQ ID NO: 2 shows the nucleotide sequence of promoter and 5′UTR region of mouse Gnat3.

SEQ ID NO: 3 shows the nucleotide sequence of promoter and 5′UTR region of human Gnat3.

SEQ ID NO: 4 shows the nucleotide sequence of promoter and 5′UTR region of cattle Gnat3.

SEQ ID NO: 5 shows a section of the amino acid sequence of mouse SLC26a8.

SEQ ID NO: 6 shows a section of the amino acid sequence of human SLC26a8.

SEQ ID NO: 7 shows a section of the amino acid sequence of pig SLC26a8.

SEQ ID NO: 8 shows a section of the amino acid sequence of goat SLC26a8.

SEQ ID NO: 9 shows a section of the amino acid sequence of cattle SLC26a8.

### DETAILED DISCLOSURE

The subject invention provides materials and methods for the prevention and/or inhibition of transmission of a particular chromosome and the generation of non-human transgenic animals of a particular sex. In some embodiments, the materials and methods of the subject invention are used to prevent and/or inhibit the transmission of a sex chromosome. In other embodiments, the materials and methods of the subject invention are used to prevent and/or inhibit the transmission of an autosome.

The prevention and/or inhibition of transmission of a particular chromosome is functionally equivalent to requiring or enforcing the transmission of the other chromosome of a chromosome pair. That is, if the transmission of a Y chromosome is prevented or inhibited it follows that the X chromosome is transmitted which is functionally equivalent to requiring the transmission of the X chromosome.

In some embodiments, materials and methods are provided for forcing transmission of a particular chromosome, wherein the particular chromosome can be a sex chromosome or an autosome.

Importantly, materials and methods of the present invention may be useful for preventing or forcing transmission of an autosome. In some embodiments, preventing transmission of a deleterious gene or allele on an autosome is achieved using materials and methods disclosed herein. In some embodiments, forcing transmission of a favorable gene or allele on an autosome is achieved using materials and methods of the present invention. In some embodiments, forcing transmission of a genetically engineered gene or allele on an autosome is achieved using materials and methods of the present invention. As used herein, a “deleterious” gene or allele refers to a gene or allele that confers harmful or injurious activities that inhibit growth and/or development of the cell or the organism. As used herein, a “favorable” gene or allele refers to a gene or allele that confers beneficial or advantageous activities that promote growth and/or development of the cell or the organism.

In specific embodiments, the subject invention provides materials and methods for producing transgenic animals, particularly non-human mammals, which transgenic animals have an altered tendency to produce progeny of a particular sex.

The term “progeny” refers to either direct offspring or descendants, i.e., offspring of offspring, depending on the sex of the animal produced.

In some embodiments, the methods of the subject invention are performed by introducing a nucleic acid construct into a chromosome of the germ line of a mammal, wherein the nucleic acid construct carries a transgene that is expressed post-meiotically in developing spermatids. Expression of the transgene is designed to alter the fertility of sperm, such that the non-human transgenic mammal has an altered tendency to produce progeny carrying the particular chromosome in a subsequent generation.

When the nucleic acid construct is introduced into a sex chromosome, the expression of the transgene can prevent and/or inhibit transmission of the respective sex chromosome to a subsequent generation.

Unlike every other cell in the body, sperm cells have a haploid genome and, thus, only an X or a Y chromosome, but not both. Thus, by inserting genes into either the X or the Y chromosomes, the fate of the respective sperm can be determined without affecting the fate of the other sperm.

Advantageously, the non-human transgenic animals of the present invention enable production of offspring of a particular sex without the need for further genetic or cell biological manipulation. For example, a male giving rise to single sex offspring can be used in natural breeding or in artificial insemination protocols. Furthermore, when a male non-human transgenic mammal is used to create the single sex offspring, the genetic modification is not passed on to subsequent generations, i.e., subsequent generations are not genetically modified.

The methods of the subject invention may be directed to producing both male and female animals. For instance, when the methods produce sperm-producing animals having the transgene on a sex chromosome, the gamete that carries the transgene after meiosis will have altered fertility, i.e., altered capability to complete fertilization of an egg. Such non-human transgenic animals will therefore have an unnatural probability of fostering progeny of a particular sex in the first generation of offspring, the probability depending on the nature of the transgene and the extent to which sperm expressing the transgene are disabled.

When the methods are used to produce egg-producing animals, the probability of having offspring of a particular sex is not affected in the first generation, because such an animal does not produce sperm. Therefore, if the transgene is on one of the two sex chromosomes, it will be passed to approximately half of the offspring depending on natural probability, whether male or female. If the transgene is on both sex chromosomes, all offspring will receive the transgene. However, the probability of having a particular sex in the first generation progeny from an egg-producing mammal will not be affected if the transgene is designed to affect sperm fertility.

Female animals that receive the transgene from a transgenic mother will carry the line, but because they do not produce sperm, their direct offspring will also not be affected. Male animals that receive the transgene, however, will have substantially single sex offspring to the extent that any sperm acquiring the transgene-bearing chromosome following meiosis is disabled. Because female animals have the capability of carrying the line indefinitely, male sperm-producers of any subsequent generation may be affected when the transgene is introduced into a line of female animals.

In some embodiments, a genetic construct of the subject invention is inserted into a Y chromosome, which genetic construct when expressed prevents and/or inhibits survival, motility, progressivity, and/or fertilization ability of the sperm carrying the respective Y chromosome. Advantageously, a non-human transgenic animal carrying said construct will not produce Y chromosome carrying sperm. The single-sexed semen of such transgenic animal can be used in natural breeding or in vitro fertilization to produce exclusively female off-spring.

In other embodiments, a genetic construct of the subject invention is inserted into a X chromosome, which genetic construct when expressed enhances or promotes, facilitates or improves survival, motility, progressivity, and/or fertilization ability of the sperm carrying the respective X chromosome. Advantageously, a non-human transgenic animal carrying said construct will have enhanced ability to produce X chromosome carrying sperm and can, thus, produce high-purity single-sexed semen to generate predominantly female off-spring.

Any technology known in the art appropriate for producing non-human transgenic animals may be used to practice the subject invention. Particularly preferred methods of producing non-human transgenic animals include, but are not limited to, spermatogonial stem cell (SCC) transfer described in U.S. Pat. No. 9,670,458, which is hereby incorporated in its entirety.

Techniques for producing non-human transgenic animals are well-known in the art and include, but are not limited to, pronuclear microinjection, viral infection, and transformation of embryonic stem cells and induced pluripotent stem (iPS) cells. Further included are techniques of site-specific knock-ins using spermatogonial stem cells, xogenous™ mobile DNA technology using transposable elements, Xanthamonas transcription activator-like nucleases (TAL-effector nucleases or TALEN), and a combination thereof. Methods of producing transgenic sperm are disclosed in U.S. Pat. No. 9,670,458.

In some embodiments, the expression construct is flanked by homology arms.

For example, targeting the transgene to either the X chromosome or the Y chromosome can be achieved by flanking the transgene with several thousand base pairs of DNA from the target X or Y chromosome. The sequence identity between the transgene construct and the X or Y chromosome promotes homologous recombination and integration of the transgene construct into the X or Y chromosome, respectively.

In certain embodiments, the exogenous nucleic acid molecule contains flanking nucleic sequences that direct site-specific homologous recombination. The use of flanking DNA sequences to permit homologous recombination into a desired genetic locus is known in the art. At present it is preferred that up to several kilobases or more of flanking DNA corresponding to the chromosomal insertion site be present in the vector on both sides of the encoding sequence (or any other sequence of this invention to be inserted into a chromosomal location by homologous recombination) to assure precise replacement of chromosomal sequences with the exogenous DNA.

Each flanking homologous arm can be from a low of about 500 base pairs (bp), about 600 bp, or about 750 bp to a high of about 2 kilo base pairs (kb), about 3 kb, or about 5kb. For example, each homologous arm can be from about 500 bp to about 1 kb, from about 500 bp to about 1.5 kb, from about 500 bp to about 2 kb, from about 500 bp to about 2.5 kb, from about 500 bp to about 3 kb, from about 500 bp to about 3.5 kb, from about 500 bp to about 4 kb, from about 500 bp to about 4.5 kb, from about 500 bp to about 5 kb, from about 600 bp to about 1.5 kb, from about 600 bp to about 2 kb, from about 600 bp to about 2.5 kb, from about 600 bp to about 3 kb, from about 5600 bp to about 3.5 kb, from about 600 np to about 4 kb, from about 600 bp to about 4.5 kb, from about 600 bp to about 5 kb, from about 750 bp to about 1.5 kb, from about 750 bp to about 2 kb, from about 750 bp to about 2.5 kb, from about 750 bp to about 3 kb, from about 750 bp to about 3.5 kb, from about 750 bp to about 4 kb, from about 750 bp to about 4.5 kb, from about 750 bp to about 5 kb.

In some embodiments, the cell may contain multiple copies of a construct of interest.

In some embodiments of the subject invention, expression constructs comprising transgenes are preferentially inserted into sex chromosomes at transcriptionally active sites. Examples of transcriptionally active sites on Y chromosomes in bovine animals, for example, include, but are not limited to, chromodomain Y like (CDY) genes, PRMAY, and members of the ZNF280BY and ZNF280AY autosome-derived Y chromosome gene families.

The materials and methods of the subject invention enable the generation of non-human transgenic animals that do not transmit a particular chromosome to off-spring.

Also provided are methods of generating non-human transgenic animals that preferentially transmit a particular chromosome to off-spring of the animals, wherein the particular chromosome can be a sex chromosome or an autosome.

In preferred embodiments, the subject invention provides materials and methods to produce non-human transgenic animals that have an altered tendency to produce progeny of a particular sex by introduction of a transgene into the germline of the animal.

In more preferred embodiments, the subject invention provides materials and methods to produce non-human transgenic animals that produce single-sexed semen. In most preferred embodiments, the materials and methods of the subject invention provide non-human transgenic animals that produce single-sexed semen that produces only female off-spring.

In preferred embodiments, the materials and methods of the subject invention create a transmission ratio distortion (TRD) in non-human transgenic animals. In specific embodiments, the TRD of the invention is accomplished by restricting the naturallY occurring transfer of RNA and proteins through cytoplasmic bridges present between spermatids during sperm development.

In preferred embodiments, the methods provided by the subject invention restrict, e.g., RNA trafficking between sperm cells. In other embodiments, the methods restrict protein trafficking between sperm using membrane insertion. In some embodiments, the methods restrict both RNA and protein trafficking between sperm cells.

In some embodiments, the trafficking of RNA and proteins between sperm cells through cytoplasmic bridges is restricted by using specific UTR tethering and/or by inserting membrane-insertion sequences into proteins.

In embodiments of the subject invention, any signal sequence that targets proteins of interest to a specific cellular location can be used to restrict the trafficking of said proteins between sperm cells.

In preferred embodiments, specific untranslated region (UTR)-derived sequences are used to tethered RNAs to cytoplasmic structures of a sperm cell.

In other preferred embodiments, the constructs of the subject invention comprise proteins that comprise membrane-insertion sequences. In some embodiments, the sequences of the invention when inserted into, e.g., a Y chromosome lead to disruption of progressivity, i.e., the ability of the sperm to find the egg; affect sperm motility, i.e., the ability of the sperm to move; or affect fertilization, i.e., the ability of the sperm to penetrate and fertilize the egg; or block survival, i.e., induce cell death in the sperm cell.

In preferred embodiments, the constructs of the subject invention express a tethered transcript in a sperm cell, which tethered transcript leads to disruption of any or all of progressivity, motility and fertilization ability in the sperm cell and/or induces sperm cell death.

In other embodiments, the expression of the tethered transcript in a sperm cell leads to facilitation, enhancement or improvement of any or all of progressivity, motility and fertilization ability in the sperm cell.

In preferred embodiments, the methods of the subject invention prevent and/or inhibit the transmission of a sex chromosome to offspring by introducing into said chromosome a construct that expresses a transcript, which transcript comprises RNA tethering UTRs that tether said transcript to a cytoskeletal structure of the sperm cell carrying the sex chromosome and restrict expression of a transgene to the sperm containing the tethered transcript.

In preferred embodiments, the nucleic acid of the tethered transcript encodes at least one protein disrupting progressivity, sperm motility, and/or the ability of the sperm to penetrate and fertilize the egg and/or induces sperm cell death.

If the transcript of the subject invention encodes at least one protein that disrupts any or all of progressivity, motility and fertilization ability and/or induces sperm cell death, the sperm containing the transcript will not be capable of fertilizing an egg and the chromosome carrying the respective transcript will not be passed to offspring.

If the transcript of the subject invention encodes at least one protein that promotes or enhances any or all of progressivity, motility or fertilization ability, the sperm containing the transcript will have improved capability of fertilizing an egg and the chromosome carrying the respective transcript will be passed to offspring.

In some embodiments, the transmission of an autosome is prevented and/or inhibited by attaching RNA tethering UTRs to a transcript expressed from an autosome, which transcript is tethered to a cytoskeletal structure and is prevented and/or inhibited from crossing cytoplasmic bridges into attached spermatids. The tethered transcript, thus, is only expressed in the sperm containing the autosome of interest.

If the cytoskeleton-tethered transcript encodes at least one protein that is disruptive to any or all of progressivity, motility or fertilization ability, or all of these in the sperm cell and/or induces sperm cell death, the sperm containing the autosome carrying the tethered transcript is disrupted in any or all of progressivity, motility or fertilization ability or will die.

If the cytoskeleton-tethered transcript encodes at least one protein that facilitates, enhances, or improves any or all progressivity, motility or fertilization ability in the sperm cell, the sperm containing the autosome carrying the tethered transcript is improved in any or all of progressivity, motility or fertilization ability.

In preferred embodiments, the tethered transcript is expressed from a Y chromosomal nucleic acid molecule. In other embodiments, the tethered transcript is expressed from an X chromosomal nucleic acid molecule.

In some embodiments of the subject invention, the protein product encoded by the transcript transcribed from the construct of the invention is prevented and/or inhibited from moving between sperm cells through cytoplasmic bridges because the protein either naturally comprises at least one membrane insertion sequence or domain or because the transcript encoding the protein has been genetically engineered such that the expressed protein comprises at least one membrane insertion sequence or domain.

In some embodiments, a construct of the subject invention expressing a transcript that encodes at least one protein comprising a membrane insertion sequence is present on a sex chromosome. In other embodiments, the construct of the subject invention expressing a transcript encoding a protein comprising a membrane insertion sequence is present on an autosome.

In further embodiments, methods are provided that use specific UTR sequences to delay translation of a protein in spermatids until the cytoplasmic bridges between spermatids are no longer present. For example, a construct of the subject invention comprises UTR sequences derived from the Smok1 gene. Smok1 is the gene encoding the t-Complex Responder (TCR) system that promotes transmission ratio distortion.

In other embodiments, the construct of the subject invention encodes at least one protein with a membrane insertion sequence, which at least one proteins leads to disruption of any or all of progressivity, motility and fertilization ability in a sperm cell or leads to sperm cell death. In these embodiments, sperm cells comprising the protein with the membrane insertion sequence are prevented from fertilizing and egg and/or inhibited to fertilize an egg and the chromosome bearing the transcript encoding for the protein with the membrane insertion sequence will not be transmitted to progeny.

In other embodiments, the construct of the subject invention encodes at least one protein comprising a membrane insertion sequence, which at least one protein leads to facilitation, enhancement and/or improvement of any or all of progressivity, motility and fertilization ability in a sperm cell. In these embodiments, sperm cells comprising the protein with the membrane insertion sequence are enhanced or improved in their ability to fertilize an egg and the chromosome bearing the transcript encoding the protein with the membrane insertion sequence will be preferentially transmitted to progeny.

In some embodiments, the protein comprising a membrane insertion sequence is a dominant negative protein that causes failure of survival, motility, and progressivity of the sperm, or failure of egg penetration by the sperm. In some embodiments, the protein comprising a membrane insertion sequence is a protein that causes failure of embryogenesis. In preferred embodiments, the dominant-negative protein is a dominant-negative form of a protein that enables and/or promotes survival, motility, progressivity and/or egg penetration of a sperm.

In preferred embodiments, the protein comprising a membrane insertion sequence is only expressed in late spermatids. In further preferred embodiments, the protein with a membrane insertion sequence is a Slc26a8 protein required for sperm motility. In some embodiments, the protein is Sept12, a microtubule complex protein required for sperm head and tail formation.

The RNA tethering UTR of the subject invention can be present on the 5′ or the 3′ side of the transgene-encoding construct, or both the 5′ and the 3′ side. The RNA tethering UTR of the subject invention can comprise any sequence that is able to tether a transcript to any membrane structure of a sperm cell and, thereby, is capable of preventing and/or inhibiting the transcript from being translocated along cytoplasmic bridges to connected sperm cells.

In preferred embodiments of the subject invention, the UTR is derived from the t-Complex Responder (TCR) encoded by the Smok gene.

In other embodiments, the UTR is derived from a Gnat3 gene.

In some embodiments, the UTR is derived from a Tas1r3 gene.

In some embodiments, the UTR is derived from a Sperm Adhesion Molecule 1 (Spam 1) gene.

In other embodiments, the UTR is derived from a Slx gene. In yet other embodiments, the UTR is derived from a Slc gene.

In yet other embodiments, the UTR is genetically engineered based on a sequence derived from any protein subject to transmission ratio distortion. The skilled artisan can readily design UTRs from different sources to be used with the materials and methods provided herein to practice the methods of the subject invention employing UTRs.

In some embodiments, the protein encoded by the UTR tethered transcript is a dominant negative protein that causes failure of survival, motility, and/or progressivity of the sperm, or failure of egg penetration by the sperm.

In some embodiments, the protein encoded by the UTR tethered transcript is a protein that causes failure of embryogenesis.

In some embodiments, the protein encoded by the UTR tethered transcript is a protein comprising a membrane insertion sequence. In some embodiments, the protein encoded by the UTR tethered transcript is a dominant negative protein that causes failure of survival, motility, and/or progressivity of the sperm, or failure of egg penetration by the sperm or a protein that causes failure of embryogenesis.

In some embodiments, the protein comprising a membrane insertion sequence contains at least one natural membrane insertion sequence. In other embodiments, the protein comprising a membrane insertion sequence contains at least one membrane insertion sequence that has been added by genetic engineering.

In some embodiments, the invention provides materials and methods to insert nucleic acid sequences on sex chromosomes, which nucleic acid sequences disrupt genes on other chromosomes.

In some embodiments, the construct of the subject invention comprises inhibitory RNA sequences that inhibit expression of at least one gene involved in survival, motility, and/or progressivity of sperm. For example, in some embodiments, small interfering RNAs (siRNAs) are generated, which siRNAs are efficient in knocking down expression of at least one gene involved in survival, motility, and/or progressivity of sperm.

In some embodiments, the construct of the subject invention comprises short-hairpin RNA (shRNA) or micro RNA (miR) sequences and the construct is inserted into a sex chromosome of a non-human animal, wherein the expression of the construct suppresses an RNA transcribed from a gene present on a non-sex chromosome.

In preferred embodiments, the short-hairpin RNA (shRNA) or micro RNA (miR) sequences target at least one gene involved in survival, motility and/or progressivity of sperm.

In some embodiments, a construct of the subject invention comprises siRNA sequences inserted into a miR cassette, which miR cassette/siRNA sequences are efficient in knocking down expression of at least one gene involved in survival, motility, and/or progressivity of sperm. In preferred embodiments, the miR cassette comprises at least one sequence that allows the introduction of the miR cassette into a 3′UTR region of a gene expressed in late spermatogenesis, thereby targeting the knock down effect to the late stage of spermiogenesis.

In some embodiments, nucleic acid construct of the subject invention comprises at least one small interfering RNA (siRNA) for at least one protein that enables progressivity, motility and/or penetration ability of a sperm cell; wherein the at least one siRNA is inserted into a micro RNA (miR) cassette, which miR cassette comprises at least one sequence homologous to a sequence of a 3′UTR region of a gene expressed in late spermatogenesis.

In preferred embodiments, the shRNAs of the construct of the subject invention target one or more genes whose products are necessary for progressivity of sperm, e.g., genes involved in the movement of sperm along chemical gradients to locate an egg. In more preferred embodiments, the shRNAs target genes encoding Gnat3 and Tas1r3 chemoreceptors. Both Tas1r3 and Gnat3 are membrane inserted proteins and are expressed very late in spermiogenesis, i.e., only in spermatids.

In some embodiments, a construct of the subject invention comprises at least one shRNA targeting the Tas1r3 gene under control of a RNA polymerase III (pol III) promoter, wherein the construct is inserted into a target chromosome to prevent and/or inhibit a sperm carrying said target chromosome from fertilizing egg and, thereby, preventing and/or inhibiting transmission of said target chromosome to offspring. In preferred embodiments, a construct of the subject invention comprises a shRNA targeting a Gnat3 gene, which construct is inserted into a target chromosome under the control of a pol III promoter to prevent and/or inhibit transmission of said target chromosome.

In some embodiments, a construct of the subject invention comprises at least one shRNA targeting a Gnat3 gene under control of a pol III promoter, which construct is inserted into a target chromosome to prevent a sperm carrying said target chromosome from fertilizing egg and, thereby, preventing and/or inhibiting transmission of said target chromosome to offspring.

In preferred embodiments, a construct of the subject invention comprises at least one shRNA targeting a Tas1r3 gene and at least one shRNA targeting a Gnat3 gene, which construct is inserted into a target chromosome under the control of a pol III promoter to prevent and/or inhibit transmission of said target chromosome.

In some embodiments, the at least one shRNA targeting Tas1r3 and the at least one shRNA targeting Gnat3 under the control of pol III promoters are located on multiple constructs.

In preferred embodiments, the at least one shRNA targeting Tas1r3 and the at least one shRNA targeting Gnat3 under the control of pol III promoters are located in a single construct.

In some embodiments, the more than one Tas1r3 shRNA units and the more than one Gant3 shRNA units are located in sequence on a construct of the subject invention.

In other embodiments, the more than one Tas1r3 shRNA units and the more than one Gant3 shRNA units are located divergently oriented to each other on a construct of the subject invention. Any groupings of multiple shRNA units on the construct are further contemplated and a skilled artisan can readily design such multiple shRNA comprising constructs.

In preferred embodiments, shRNAs can be present in any multimer, including, but not limited to, one, two, three or more shRNA targeting multimers on the construct of the subject invention.

In some embodiments, the shRNA units are separated by terminator sequences, especially in constructs that comprise multiple shRNA units located in sequence, i.e., transcribed in the same direction.

In other embodiments, where the shRNA units are oriented divergent to each, i.e., where the 3′ ends of each shRNA unit face each other, terminator sequences are optional.

In certain embodiments, the constructs comprise multiple cloning sites between the several shRNA units and/or at the 5′ and 3′ end of the construct.

In certain embodiments, the pol III promoters include, but are not limited to, U6 promoters and H1 promoters.

In some embodiments, a genetic construct comprising at least one shRNA targeting the Tas1r3 gene and/or at least one shRNA targeting Gnat3 under the control of pol III promoters are inserted into a sex chromosome to prevent and/or inhibit transmission of said sex chromosome to offspring.

In other embodiments, a genetic construct comprising at least one shRNA targeting the Tas1r3 gene and/or at least one shRNA targeting Gnat3 under the control of pol III promoters are inserted into an autosome to prevent and/or inhibit transmission of said autosome to offspring.

In preferred embodiments, the genetic construct comprising at least one shRNA targeting the Tas1r3 gene and/or at least one shRNA targeting Gnat3 under the control of pol III promoters are inserted into a Y chromosome to prevent and/or inhibit transmission of said Y chromosome to offspring, thereby generating non-human transgenic animals that only produce semen of a single sex, i.e., only semen to father female offspring.

Advantageously, non-human transgenic animals producing single-sexed semen produced using the materials and methods of the subject invention do not pass the transgene to their offspring, i.e., the offspring is not genetically modified and the production of single-sexed offspring using such non-human transgenic animal of the subject invention does not involve any further genetic or cell biological manipulations but offspring can be obtained through natural breeding techniques.

In some embodiments the constructs comprising at least one Tas1r3 shRNA and/or at least one Gnat3 shRNA do not contain RNA tethering or protein insertion sequences. In such embodiments, the RNAs and proteins expressed from the construct can be exchanged between sperm cells connected through cytoplasmic bridges and sperm cells carrying the construct as well as those not carrying the construct can be negatively affected by the RNA and protein expressed from construct.

The subject invention further provides materials and methods to rescue sperm cells that carry a construct lacking RNA tethering or protein insertion sequence by inserting an expression cassette using a Tas1R3 and/or Gnat3 gene under their native promoters but with the “3^{rd} bases” wobbled so the shRNAs expressed from the same construct no longer recognize the Tas1r3 and Gnat3 gene sequences. In these embodiments, only those sperm cells carrying the construct are viable because the wobbled Tas1r3 and/or Gnat3 genes are immune to suppression by the co-expressed shRNAs against Tas1r3 and/or Gnat3.

In some embodiments, a nucleic acid molecule is provided comprising at least one short hairpin RNA (shRNA) for a protein that enables progressivity, motility, or penetration ability of a sperm cell; wherein the at least one shRNA is operably linked to a pol III promoter selected from a U6 promoter and a H1 promoter.

In further embodiments, the at least one shRNA is against aTas1R3 and/or Gnat3 protein.

In preferred embodiments, the nucleic acid further comprises an 22xogenous nucleic acid sequence encoding a Gnat3 protein operably linked to a Gnat3 promoter, wherein the nucleic acid sequence encoding the Gnat3 protein comprises third base wobbles such that the shRNA against Gnat3 does not bind said nucleic acid sequence encoding the Gnat3 protein.

In further preferred embodiments, the exogenous Gnat3 sequence comprises a UTR tethered transcript and/or the encoded Gnat3 protein contains a protein membrane insertion sequence to restrict the rescue to those sperm cells that carry the exogenous construct of the subject invention.

In some embodiments, a construct of the subject invention comprises a “wobbled” Gnat3 and/or a “wobbled ” Tas1r3 gene and further comprises at least one pol III promoter driven Tas1r3 shRNA and/or at least one Gnat3 shRNA arranged on the construct either sequentially with terminator sequences between shRNA units or divergently with or without terminator sequences between shRNA units.

Advantageously, non-human transgenic animals generated using such shRNA/wobbled constructs of the subject invention express a Tas1r3 and/or Gnat3 protein from the sperm cell containing the “wobbled” Tas1r3 and/or Gnat3 genes and such sperm cells are able to fertilize an egg.

In contrast, sperm cells not containing the construct containing the wobbled Tas1r3 and/or Gnat3 genes and only containing the Tas1r3 shRNAs and/or Gnat3 shRNAs through cytoplasmic bridges will undergo inhibition of endogenous Tas1r3 and/or Gnat3 mRNA expression and will be unable to fertilize an egg.

In some embodiments, shRNAs to Sept-4 and/or shRNAs to Sept12 are inserted into a construct of the subject invention.

In some embodiments, shRNAs to CATSPER1 to CATSPER4 are inserted into a construct of the subject invention.

In a further embodiment, shRNAs to CATSPERB, CATSPERD, and CATSPERG are inserted into a construct of the subject invention.

In some embodiments, non-human transgenic animals of the subject invention comprise a construct of the invention on an autosome, which autosome, e.g, carries an undesirable mutated allele, and such construct-bearing autosome-containing sperm will not be transmitted to offspring if the construct comprises a transgene containing either a UTR tethered transcript or encoding a protein with a membrane insertion sequence and the transgene encoded protein leads to disruption of any or all of progressivity, motility or fertilization ability in the sperm cell or induces sperm cell death. Advantageously, male non-human transgenic animals produced using such construct will only father offspring that do not contain the undesirable mutant allele.

Further, the introduction of the genetic construct of the subject invention into a selected autosome containing a mutant allele can be achieved by providing the mutant allele sequence in one of the homologous arms flanking the construct to be inserted into the autosome. The skilled artisan can determine, based on the size and characteristic of the mutation(s) on the undesirable allele, the length and content of the homologous arms used for homologous recombination and insertion of the construct at or near the location of the mutant allele of the autosome. Thus, based on e.g., the teachings of U.S. Pat. No. 9,670,458, which is incorporated by reference, will readily recognize how to design the homologous arms flanking the construct sequence.

Advantageously, the basic concepts of the subject invention are applicable to a variety of applications based on a variety of desirable and undesirable characteristics and traits expressed on one, but not the other, autosome of an autosome pair.

For example, any characteristic or trait differentially expressed in one of a pair of autosomes can be used in the methods of the subject invention to express an UTR tethered transcript containing transcript and/or a cytoskeleton-tethered protein in the sperm cells containing said target autosome where the tethered transcript and/or tethered protein when expressed in the sperm cells cause failure of sperm cell survival, motility, and/or progressivity of the sperm, or failure of egg penetration by the sperm cell, thus, preventing and/or inhibiting transmission of the undesirable characteristic or trait to the offspring.

The targeting of the tethering UTR-containing transcript to a sex chromosome or an autosome can be achieved by homologous recombination techniques and/or gene editing techniques known in the art. The person with skill in the art of homologous recombination techniques and gene editing techniques readily recognizes the requirement for a threshold number of nucleotides distinct between an undesirable target allele and a wild-type allele in order to enable specific targeting of said target allele by a construct of the subject invention. The methods of the subject invention can, therefore, be used to replace or edit traits including, but are not limited to, traits caused by deletions, insertions, or multi-nucleotide mutations.

Methods for introducing exogenous nucleic acid molecules into sex chromosomes of an animal are known in the art. Genes specifically located in the X and Y chromosome have previously been identified (see, e.g., U.S. Pat. Nos. 5,595,189; 5,700,926; and 5,763,166, incorporated herein by reference).

In preferred embodiments, the subject invention provides methods to insert genetic constructs into a sex chromosome of an animal which inserted construct comprises at least one gene that can destroy a sperm cell containing the target sex chromosome sperm through, e.g., induction of apoptosis.

To affect specific expression of the transgene in developing spermatids, expression of the transgene must be controlled by a sperm-specific control sequence. Such control sequence may affect specific expression in sperm either by transcriptional or translational control mechanisms.

In preferred embodiments, the control sequence is a sperm cell-specific promoter that specifically affects transcription only in post-meiotic spermatids. Many such promoters have been identified, any of which may be used in the subject invention to practice the methods of the invention and affect specific expression of the transgene in post-meiotic sperm.

The promoter used in the subject invention can be any promoter active in late spermatogenesis, but preferentially a promoter with strong expression only in late spermatogenesis, to avoid effects in other tissues. Thus, any promoter of a gene specific to postmeiotic sperm can be used.

In preferred embodiments, the promoter is a promoter of a strongly expressed gene specific to the acrosome, flagella, or late-expressing flagella motors. For example, appropriate promoters to practice the subject invention include, but are not limited to, promoters of the sperm mitochondrial maintenance gene Spatal9 promoter, the outer dense fiber of sperm tails 3b (Odf3b) promoter, the outer dense fiber of sperm tail 1(Odf1) promoter, the outer dense fiber of sperm tail 3 (Odf3) promoter, the protamine promoter, the TNP-1 promoter, the sperm mitochondria associated cysteine rich protein (smcp) promoter, the testis specific promoter within the sixteenth intron of the cKIT gene, the taste receptor type 1 member 3 (Tas1r3) promoter, the gustducin alpha-3 chain (Gnat3) promoter, and any other promoter regulating the expression of a gene that is specific to the acrosome, flagella or late-expressing flagella motors and/or is strongly expressed in post-meiotic sperm. Furthermore, the skilled artisan can readily recognize that promoters to be described in the future art based can be used to practice the subject invention based on the instant disclosures of the requirements for such promoters.

In some embodiments, the promoters that drive expression of apoptosis-inducing genes in sperm containing the target chromosome are promoters that are active in late stages of spermatogenesis when the physical interconnection between spermatocytes has subsided and the effects of the expression of apoptosis-inducing genes are limited to the sperm cells in which the respective apoptosis-inducing genes are expressed.

In some embodiments, when the method of the subject invention comprises immobilizing sperm containing the unwanted chromosome, similar promoters as enumerated above can be used. However, because low expression and expression restricted to late stages of sperm development are less important in these embodiments, promoters can be used that are active in mature spermatogonia, including universal promoters.

In some embodiments, the universal promoters useful in such embodiment of the subject invention include, but not limited to, cytomegalovirus (CMV) promoter, CMV-chicken beta actin promoter, ubiquitin promoter, JeT promoter, SV40 promoter, beta globin promoter, elongation Factor 1 alpha (EF1-alpha) promoter, Mo-MLV-LTR promoter, Rosa26 promoter, and any combination thereof. It is within the purview of the skilled artisan to determine experimentally the optimal promoter to be used to practice the methods of the subject invention based on the teachings of the instant application and the disclosed requirements for promoter functionality during specific stages of sperm development. Thus, any additional promoter identified in the art as being active during specific stages of sperm development can be used to practice the subject invention and is within the purview of the skilled artisan.

In most preferred embodiments, the promoters used in the methods of the subject invention are sperm cell-specific promoters that are highly active in spermatogonia, not or minimally active in earlier stages of sperm development, and inactive in any other tissue throughout the body.

The proteins expressed in sperm cells from constructs of the subject invention including UTR tethered transcript constructs and/or constructs comprising proteins with a membrane insertion sequence include any protein that causes asthenozoospermia in a mammal.

Proteins causing asthenozoospermia and useful for the subject invention include, but are not limited to, mutant forms of SUNS, several septins, including Sept4 and Sept12, cation channel sperm associated (CATSPER) mutations, including mutant forms of CATSPER1 and CATSPER2, mutant anion transporter SLC26A8, mutant Spata16, mutant PLCZ1, mutant DPY19L2, mutant Gpx4, mutant Hook1, mutant Prrs21, mutant Oaz3, mutant Cntrob, mutant Ift88. The proteins useful to practice the subject invention either have endogenous membrane insertion sequences or are genetically engineered to have membrane-inserting sequences.

Further proteins useful for the subject invention include Ubiquitin specific peptidase 9, Y linked (USP9Y), Dead box on Y (DBY), Ubiquitously transcribed tetratricopeptide repeat gene, Y linked (UTY), lysine-specific demethylase 5D (KDM5D), eukaryotic translation initiation factor 1A, Y linked (EIF1AY), Ribosomal protein S4 Y isoform 2 (RPSAY2), Chromosome Y open reading frame 15A and 15B (CYORF15A and CYORF15B), XK, Kell blood groups complex subunit-related, Y linked (XKRY), Heat shock transcription factor, Y linked (HSFY), RNA binding motif protein, Y linked (RBMY1), PTPN13-like, Y linked (PRY), Chromodomain Y, Y linked (CDY), Basic protein Y2, Y linked (BPY2), Deleted in azoospermia (DAZ), Chondroitin sulfate proteoglycan 4-like, Y0linked pseudogene 1 (CSPG4LYP1), and Golgi autoantigen, golgin subfamily a2-like, Y linked 1 (GOLGA2LY1).

In a preferred embodiment, the transgene of the subject invention is a dominant negative mutant gene that encodes for an altered gene product that acts antagonistically to the wild-type allele. For example, the dominant negative mutant gene of the subject invention can be a dominant negative SUNS, a dominant negative mutant Sept4, a dominant negative Sept12, a dominant negative CATSPER1, a dominant negative CATSPER2, a dominant negative SLC26A8, a dominant negative Spata16, a dominant negative PLCZ1, a dominant negative DPY19L2, and/or a dominant negative form of Gpx4, a dominant negative form of Hook1, a dominant negative form of Prrs21, a dominant negative form of Oaz3, a dominant negative form of Cntrob, a dominant negative form of Ift88.

Useful for the practice of the methods of the subject invention is any gene involved in apoptosis, including genes that have been developed to induce apoptosis by administering an activating agent.

In some embodiments, a method of the subject invention comprises expressing a transgene on an undesirable sex chromosome to force the sperm cell containing said undesired sex chromosome into cell suicide or programmed cell death. Suicide transgenes suitable to practice the subject methods include, but are not limited to, Herpes virus-thymidine kinase/acyclovir or ganciclovir system, a cytosine deaminase/5-fluorocytosine, a cytosine deaminase/uracil phosphoribosyltransferase system, a Varicella-Zoster thymidine kinase system, purine nucleoside phosphorylase (PNP) system, carboxypeptidase A and carboxypeptidase G2 systems, beta-galactosidase system, nitroreductase system, hepatic cytochrome P450-2B1 system, a modified CYP4VB1 protein system, a dominant-negative MYC-interfering protein system, an alkaline phosphatase system, penicillin-V amidase system, thymidylate kinase/azidothymidine system, caspase-1, caspase-3, capsase-6, casase-8, and caspase-9 systems. This list is only exemplary and any suicide gene developed for the induction of apoptosis in target cells can be useful in the practice of the methods of the subject invention.

In further embodiments, other elements to enhance transcription, translation, and/or selection, e.g., introns, polyadenylation sequences, marker sets, etc., can be present in the transgene constructs of the subject invention. The person with skill in the art can readily recognize the advantageous function of these elements and can readily include the respective elements in the constructs of the subject invention.

In preferred embodiments the subject invention provides non-human transgenic animals that can be used to generate single-sexed semen.

Single-sexed semen, as used herein, means that a semen preparation is composed of at least 80% of sperm cells that contain a desired sex chromosome. For example, the single-sexed semen can be composed of at least 80% of sperm cells containing an X chromosome or the single-sexed semen can be composed of at least 80% sperm cells containing a Y chromosome. The single-sexed semen can contain a low of about 80% of sperm cells containing the single, desired sex chromosome to a high of about 100% of sperm cells containing the single, desired sex chromosome. Furthermore, the single-sexed semen can contain from about 81% to about 99%; from about 82% to about 98%; from about 83% to about 97%, from about 84% to about 96%, from about 85% to about 95%, from about 86% to about 94%, from about 87% to about 93%, from about 88% to about 92%m from about 89% to about 91% of sperm cells containing the single, desired sex chromosome.

In preferred embodiments, the subject invention provides non-human transgenic animals that produce single-sexed semen, i.e., semen that comprises at least 80% of X chromosome-containing sperm cells or at least 80% of Y chromosome-containing sperm cells.

In specific embodiments, the subject invention provides materials and methods for the production of high purity sperm lacking a particular chromosome.

The use of the term “purity” or “high purity” should be understood to be the percentage of the isolated sperm cell population containing a particular differentiating characteristic or desired combination of characteristics. For example, where a population of sperm cells is separated based on containing an X chromosome as opposed to a Y chromosome, an X chromosome containing population of sperm cells having at least 60% purity comprises a population of sperm cells of which at least 60% of the individual sperm cells contain an X chromosome while 40% of the sperm cell population contain a Y chromosome.

A high-purity semen composition can have from a low of about 60% to a high of about 79% of sperm cells containing the single-desired sex chromosome. For example, a high-purity semen composition can have from about 61% to about 78%; from about 62% to about 77%; from about 63% to about 76%; from about 64% to about 75%; from about 65% to about 74%; from about 66% to about 73%; from about 67% to about 74%; from about 68% to about 73%; from about 69% to about 72%; from about 70% to about 71% of sperm cells containing the single, desired sex chromosome.

The semen produced using the materials and methods of the subject invention can be used to fertilize oocytes either during natural breeding, artificial insemination of a female, in vitro fertilization of oocytes, or intracytoplasmic injection of sperm cells, or the like to produce progeny. The term “progeny” refers to either direct offspring or descendants, i.e., offspring of offspring.

The sperm cells produced by the methods of the subject invention can include sperm cells from a male of any species of mammal including, but not limited to, sperm cells from humans, and animals such as bovids, equids, ovids, canids, felids, goats, swine, primates as well as less commonly known mammals such as elephants, deer, zebra, camels, or kudu. This list of animals is intended to be exemplary of the great variety of animals from which sperm cells can be routinely obtained.

As used herein, the term “expression construct” refers to a combination of nucleic acid sequences that provides for transcription of an operably linked nucleic acid sequence. Expression constructs of the subject invention also generally include regulatory elements that are functional in the intended host cell in which the expression construct is to be expressed. Thus, a person of ordinary skill in the art can select regulatory elements for use in, for example, bacterial host cells, yeast host cells, plant host cells, insect host cells, mammalian host cells, and human host cells. Regulatory elements include promoters, transcription termination sequences, translation termination sequences, enhancers, and polyadenylation elements.

As used herein, the term “operably linked” refers to a juxtaposition of the components described wherein the components are in a relationship that permits them to function in their intended manner. In general, operably linked components are in contiguous relation. Sequence(s) operablY linked to a coding sequence may be capable of effecting the replication, transcription and/or translation of the coding sequence. For example, a coding sequence is operablY linked to a promoter when the promoter is capable of directing transcription of that coding sequence.

A “coding sequence” or “coding region” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus.

The term “promoter,” as used herein, refers to a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

In addition to a promoter, one or more enhancer sequences may be included such as, but not limited to, cytomegalovirus (CMV) early enhancer element and an SV40 enhancer element. Additional included sequences are an intron sequence such as the beta globin intron or a generic intron, a transcription termination sequence, and an mRNA polyadenylation (pA) sequence such as, but not limited to, SV40-pA, beta-globin-pA, the human growth hormone (hGH) pA and SCF-pA.

The term “divergent orientation” of promoters, as used herein, refers to the location of two or more promoters on a nucleic acid molecule such that transcription initiated from each promoter proceeds in opposite directions on the nucleic acid molecule. Synonyms for divergently oriented are divergently coupled promoters, and promoters oriented in opposite directions.

The term “polyA” or “p(A)” or “pA” refers to nucleic acid sequences that signal for transcription termination and mRNA polyadenylation. The polyA sequence is characterized by the hexanucleotide motif AAUAAA. Commonly used polyadenylation signals are the SV40 pA, the human growth hormone (hGH) pA, the beta-actin pA, and beta-globin pA. The sequences can range in length from 32 to 450 bp. Multiple pA signals may be used.

The term “nucleic acid” as used herein refers to RNA or DNA molecules having more than one nucleotide in any form including single-stranded, double-stranded, oligonucleotide or polynucleotide.

The term “nucleotide sequence” is used to refer to the ordering of nucleotides in an oligonucleotide or polynucleotide in a single-stranded form of nucleic acid.

The term “expressed” refers to transcription of a nucleic acid sequence to produce a corresponding mRNA and/or translation of the mRNA to produce the corresponding protein. Expression constructs of the subject invention can be generated recombinantly or synthetically or by DNA synthesis using well-known methodology.

The term “regulatory element” as used herein refers to a nucleotide sequence which controls some aspect of the expression of an operably linked nucleic acid sequence.

Exemplary regulatory elements illustratively include an enhancer, an internal ribosome entry site (IRES), an intron, an origin of replication, a polyadenylation signal (pA), a promoter, a transcription termination sequence, and an upstream regulatory domain, which contribute to the replication, transcription, post-transcriptional processing of a nucleic acid sequence. Those of ordinary skill in the art are capable of selecting and using these and other regulatory elements in an expression construct with no more than routine experimentation.

In one embodiment, the construct of the present invention comprises an internal ribosome entry site (IRES). In one embodiment, the expression construct comprises kozak consensus sequences.

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms. The terms “nucleic acid” or “nucleic acid sequence” encompass an oligonucleotide, nucleotide, polynucleotide, or a fragment of any of these, DNA or RNA of genomic or synthetic origin, which may be single-stranded or double-stranded and may represent a sense or antisense strand, peptide nucleic acid (PNA), or any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

The term “wobbled” gene sequence refers generally to a nucleic acid sequence that has been changed only in the third base of an amino acid encoding trinucleotide such that the amino acid encoded by the trinucleotide is not changed and, thus, the encoded protein sequence is not changed, but the protein encoding nucleic acid sequence is different. The exchange of the third nucleotide of the amino acid encoding nucleic acid sequence can prevent and/or inhibit the binding of, e.g., a siRNA and/or shRNA to the nucleic acid sequence and can, thus, prevent and/or inhibit siRNA and/or shRNA mediated suppression of gene expression.

The terms “membrane insertion sequence” or “membrane insertion domain” refers generally to a protein sequence or domain that aids in insertion of a protein or a part of a protein into a cellular membrane, wherein the cellular membrane can be the plasma membrane or a membrane of an intracellular organelle. It is within the purview of a person with ordinary skill in the art to determine which protein sequences are membrane insertion sequences and, thus, useful for the practice of the subject invention.

The term “untranslated region” or UTR refers generally to any nucleic acid sequence that is not translated into a protein. In the context of the constructs of the invention, the UTR includes a UTR that mediates RNA tethering to cytoskeletal structures of a cell. For example, a UTR of the invention can tether a transcript to any cytoskeletal structure of the cell in which the UTR-containing transcript is present. It is within the purview of a person with ordinary skill in the art to determine which UTR sequences tether a transcript to cytoskeletal structures of a cell and to which cytoskeletal structures said UTRs tether a transcript. Any UTR tethering any transcript to any cytoskeletal structure present in a sperm cell are useful for the practice of the subject invention.

### EXAMPLES

### Example 1: Determination of siRNAs to Suppress TAS1R3 and GNAT3 Expression

Mice that are heterozygous for loss of TAS1R3 and GNAT3, which are located on mouse chromosomes 4 and 5 respectively, never pass along sperm missing both genes to their offspring. These genes are required for sperm progressivity, i.e., finding the egg. This indicates that the RNA for these genes is not shared between developing sperm through cytoplasmic bridges or intracellular channels, as most RNA is, and the resulting proteins are not shared between developing sperm, as most proteins are.

In order to achieve the goal of a transgenic animal in which an arbitrary chromosome is not transmitted, TAS1R3 and GNAT3 must be suppressed using genetic constructs inserted elsewhere on said chromosome. In general, suppression of genes can use a dominant negative approach, or siRNA. Effective siRNA for TAS1R3 and GNAT3 were determined using standard techniques known in the art and constructs comprising TA1R3 and GNAT3 siRNA under a POL III promoter were created. These mice were used to determine the prevention and/or inhibition of transmission of the chromosome carrying the construct.

### Example 2: Gnat3/Tas1r3 Double Knockout Mice

In order to determine the effect of the taste chemoreceptors, Gnat3 and Tas1r3, on transmission ratio distortion, mice having one or both of Gant3 and Tas1r3 genes knocked out have previously been generated ¹. Table 1 shows the effects of single and double-knock outs on female and male transmission¹. While the predicted and observed percentages of transmission were roughly the same in female transmission, double-knock out of Gnat3 and Tas1r3 resulted in 0% male transmission in view of a predicted male transmission of 25% and 50% in the different cross breedings. These results confirmed that sperm lacking both Gnat3 and Tas1r3 are unable to find the egg and, thus, incapable of fertilization.

TABLE 1
Transmission Ratio Distortion

| | Female transmission | | Male transmission | |
|---|---|---|---|---|
| | Observed | Predicted | Observed | Predicted |
| Donor haplotypes | (%) | (%) | (%) | (%) |
| Cross 1 | | | | |
| Gnat3+ Tas1r3− | 45 | 50 | 100 | 50 |
| Gnat3− Tas1r3− | 55 | 50 | 0 | 50 |
| Cross 2 | | | | |
| Gnat3+ Tas1r3+ | 24 | 25 | 32 | 25 |
| Gnat3− Tas1r3+ | 24 | 25 | 33 | 25 |
| Gnat3+ Tas1r3− | 25 | 25 | 35 | 25 |
| Gnat3− Tas1r3− | 27 | 25 | 0 | 25 |

Reproduced from (1).

### Example 3: Pol III Promoter Tas1r3/Gnat3 shRNA Progressivity Transmission Ratio Distortion (TRD) Mice

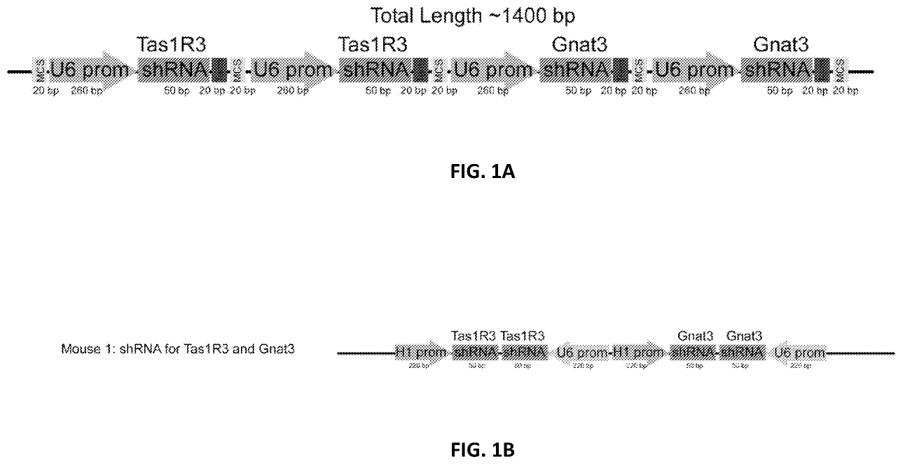

To prevent and/or inhibit transmission of an arbitrary chromosome, genetic constructs were designed that knock- down expression of either the Tas1R3 gene or Gnat3 gene or both, which constructs can be inserted into any chromosome and will prevent and/or inhibit the successful fertilization of a sperm carrying the chromosome with the genetic construct. In one example, the construct comprises U6 pol III promoters driving shRNA for Tas1R3 and Gnat3. The pol III promoters can include, but are not limited to, U6 promoters and H1 promoters. Preferably, two shRNAs for each of Tas1R3 and Gnat3 are used in a single construct in order to ensure >90% knockdown. The use of shRNA expressed from a U6 promoter is known in the art and has been shown to be successfully in live mice. Importantly, it is also known in the art that shRNAs are still functional in spermiogenesis. A first construct was generated that comprises sequentially arranged promoter/shRNA units comprising two units of a U6 promoter operably linked to a Tas1R3 shRNA and a terminator and two units of a U6 promoter operably linked to a Gnat3 shRNA and a terminator with multiple cloning sites located between each U6 promoter/shRNA unit and a multiple cloning site at each end of the construct (FIG. 1A). This construct can also be generated using H1 promoters to replace at least one of the U6 promoters or to replace all U6 promoters.

A second construct was generated that comprises divergently oriented promoter/shRNA units of U6 promoters or H1 promoters operably linked to Tas1R3 shRNAs and Gnat3 shRNAs (FIG. 1B). In this construct, the two Tas1R3 shRNAs are operably linked to U6 or H1 promoters, respectively, such that transcriptions from the U6 or H1 promoters proceed towards each other and the Tas1R3 shRNAs function as each other's terminator sequence. Similarly, the two Gnat3 shRNAs are operably linked to U6 or H1 promoters, respectively, such that transcriptions from the U6 or H1 promoters proceed towards each other and the Gnat3 shRNAs function as each other's terminator sequence.

The insertion of the described constructs into an arbitrary chromosome allows the prevention and/or inhibition of a fertilization event mediated by a sperm containing such chromosome and, thus prevents and/or inhibits the transmission of said chromosome to offspring.

The construct from FIG. 1B was used to create transgenic mice via pronuclear injection on an FVB/N strain background, with the construct inserted into a single random autosome. A total of 66 mice offspring were born live from multiple litters from multiple wild-type females bred to a single male founder. Results showed that 62 out of the 66 offspring were negative for the chromosome targeted for transmission prevention, demonstrating a 94% (62/66) transmission ratio distortion (TRD, Table 2). Testing for transgene was performed using two quantitative PCR primer sets specific for the insert, and a control set for genomic DNA to demonstrate that a negative result was not due to lack of DNA or failure of PCR.

TABLE 2
Transmission Ratio Distortion of Pol III Promoter
Tas1r3/Gnat3 shRNA Progressivity Mice Offspring

| Category | Observed | Expected |
|---|---|---|
| Negative | 62 | 33 |
| Positive | 4 | 33 |
| Total | 66 | 66 |

Transmission Ratio Distortion (TRD) = 94%

Taken together, the results indicate that Tas1r3/Gnat3 shRNA in the parent transgenic mice successfully prevented transmission of the targeted chromosome to the offspring. The slight imperfection (94% rather than 100%) is likely because of random insertion site, rather than inherent to the methodology.

### Example 4: Rescued Pol III Promoter Tas1r3/Gnat3 shRNA Progressivity Transmission Ratio Distortion (TRD) Mice

To prevent and/or inhibit any effects related to shRNAs crossing the cytoplasmic bridges, non-human transgenic animals are generated that comprise a genetic construct comprising shRNAs, e.g., for Tas1r3 and Gnat3 or both and additionally comprise a rescue element that comprises a Tas1r3 and/or Gnat3 gene made resistant to the respective shRNAs by introducing 3^{rd} base wobbles into the coding sequence of the Tas1r3 and/or Gnat3 genes.

Therefore, rescue of Tas1R3 and/or Gnat3 can be accomplished by inserting an expression cassette using a Tas1R3 or Gnat3 gene under their native promoters but with the 3^{rd} bases wobbled so the shRNA no longer recognizes the Tas1R3 and Gnat3 gene sequences. In this case, only those sperm carrying the construct will be viable because the wobbled Tas1R3 and Gnat3 gene are immune to suppression by the shRNA.

In one genetic construct, Gnat3 was used because the promoter and 5′ UTR of Gnat3 are well conserved across species with ˜80% identity between mice, humans, and cattle.

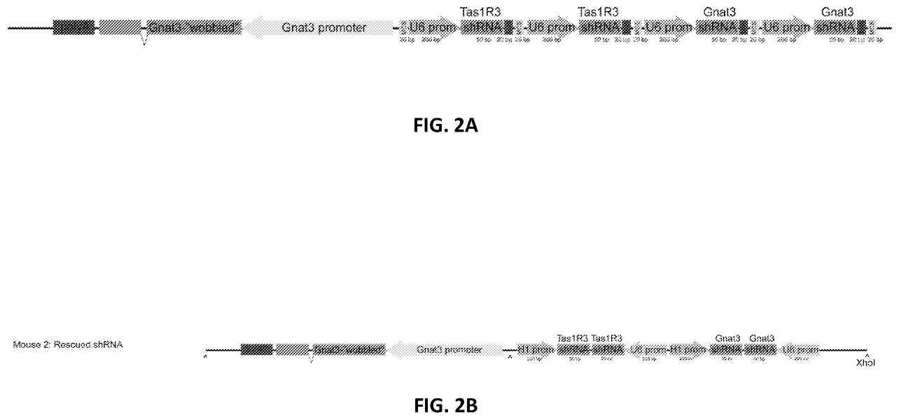

The genetic construct comprising a nucleic acid sequence encoding a Gnat3 mRNA in which the nucleotides comprising the binding sites of the shRNA on the endogenous Gnat3 mRNA sequence have been changed to prevent and/or inhibit the exogenous Gnat3 shRNA from binding and inhibiting the co-expressed exogenous Gnat3 mRNA is shown in FIG. 2A. In this system, the Gnat3 shRNA only binds to and inhibits the endogenous Gnat3 mRNA but cannot bind or inhibit the exogenously added wobbled Gnat 3 mRNA. The exogenous Gnat3 sequence comprises a RNA tethering UTR and the encoded Gnat3 protein contains a protein membrane insertion sequence to restrict the rescue to those sperm cells that carry the exogenous construct.

A construct was generated that comprises a Gnat3 promoter operably linked to a wobbled Gnat3 gene and a polyA sequence (FIG. 2A). Another construct was generated that comprises a Gnat3 promoter operably linked to a wobbled Gnat3 gene and a SV40 polyA sequence (FIG. 2B).

Each of the Gnat3 promoter-wobbled Gnat3 gene constructs are either combined with the sequentially arranged U6 promoter/Tas1R3 shRNA and U6 promoter/Gnat3 shRNA construct of FIG. 1A or the divergently linked U6 or H1 promoter/Tas1R3 shRNA and U6 or H1 promoter/Gnat3 shRNA construct of FIG. 1B.

Advantageously, transgenic mice generated using the constructs of FIGS. 2A and 2B will express a Gnat3 protein from the construct comprising the wobbled Gnat3 gene because the Gnat3 shRNA co-expressed from the combined construct cannot bind the mRNA transcribed from the wobbled Gnat3 gene sequence and, thus, does not inhibit expression of the Gnat3 protein encoded by the wobbled Gnat3 gene.

The Pol III promoters of the constructs of the invention including, but not limited to, the U6 and H1 promoters are interchangeable such that each of the U6 promoters in the constructs of the figures can be replaced with, e.g., a H1 promoter and each of the H1 promoters can be replaced with, e.g., a U6 promoter. Furthermore, the constructs can contain multiple cloning sites between each of the promoter/shRNA elements and between the promoter/shRNA elements and the Gnat3 promoter-wobbled Gnat3 element.

In addition, the 3′end of the Gnat3 gene can be preserved to maintain small introns present therein for improved translation.

### Example 5: Mice with Recombination Sites Inserted into the Y Chromosome

To insert genetic constructs into the Y chromosome, constructs were generated that introduce specific recombination sites into a mouse genome at desired sites in the Y chromosome. These mice can be used to introduce through pronuclear injection any of the constructs of the subject invention together with integrase and create an animal in which the Y chromosome specifically is not transmitted.

The technology for rapid site-specific integration is known in the art and is provided commercially, e.g., by Applied StemCell.

The site of integration into the Y chromosomes is selected based on the following criteria: (1) transcriptional activity of the site, i.e., open chromatin during the one-cell embryo stage and (2) transcriptional activity, i.e., open chromatin during late spermatogenesis.

One site used is the site near Dby, also known as Ddx3y, RNA of which gene is found in high abundance in male blastocysts and in spermatogonia. Importantly, Dby is only expressed in male, not female, blastocysts, thus, Dyb is unlikely carried to the blastocysts by sperm.

### Example 6: Gnat3 Promoter and UTR Combination with a SLC26a8 Dominant Negative Gene

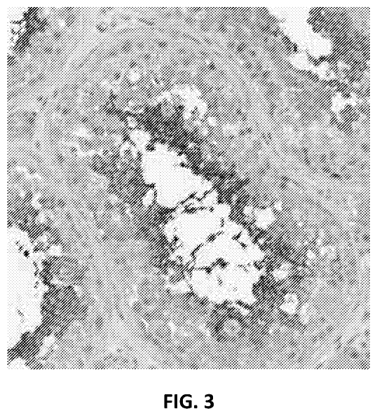

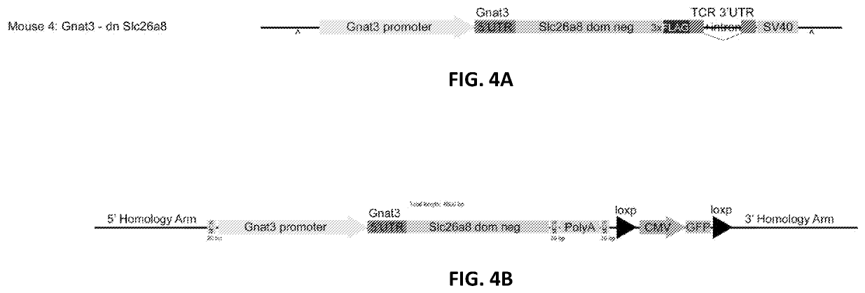

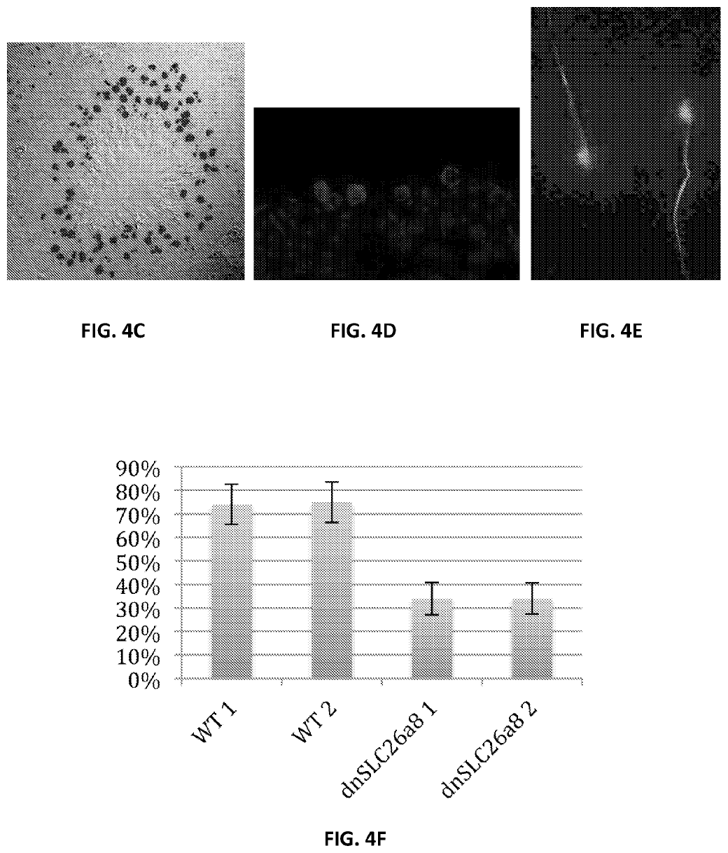

Slc26a8 is a required co-factor for Cystic Fibrosis Conductance Regulator (CFTR) in sperm, but not other sites of CFTR expression. Slc26a8 is a membrane-inserted protein only expressed in late spermatids and is required for sperm motility (2) (see, e.g., FIG. 3, reproduced from (2)). In the absence of Slc26a8, sperm is unable to move because of problems in energy production. Dominant negative Slc26a8 is known to cause infertility in humans. Constructs were generated comprising a Gnat3 promoter operably linked to the Gnat3 5′ UTR, a Slc28a8 dominant negative gene, and a t-complex responder (TCR) 3′UTR which contains an intron followed by the SV40 polyA (FIG. 4A). A further construct was generated comprising a Gnat3 promoter operably linked to the Gnat3 5′ UTR, a Slc28a8 dominant negative gene, and a polyA followed by loxP sites surrounding a CMV promoter-GFP cassette and the entire construct was embedded between homologous arms 5′ and 3′ of the construct to enable introduction of the construct into a chromosome to generate a non-human transgenic animal (FIG. 4B). The inclusion of loxP sites surrounding the CMV promoter-GFP cassette allows removal of this cassette by administration of a Cre recombinase as known in the art.

The construct from FIG. 4A was used to create transgenic mice via pronuclear injection on an FVB/N strain background, with the construct inserted into a single random autosome. A total of 17 mice offspring were born live from two litters from two wild-type females bred to a single male founder. Results showed that 16 out of the 17 offspring were negative for the chromosome targeted for prevention of transmission, demonstrating a 94% (16/17) transmission ratio distortion (TRD, Table 3). Testing for transgene was performed using two quantitative PCR primer sets specific for the construct, and a control set for genomic DNA to demonstrate that a negative result was not due to lack of DNA or failure of PCR. The Gnat3 RNA tethering is shown in FIG. 4C, and the SLC26a8 protein tethering is shown in FIGS. 4D-E. RNA tethering in FIG. 4C was detected using RNAScope using probes specific to the RNA produced by the construct (the probes matched and hybridized only to the construct, not to endogenous SLC26a8). Protein tethering in FIG. 4D was detected using the 3× flag attached to the end of the construct protein; this also shows localization within the mature sperm in FIG. 4E, localizing to the midpiece kink characteristic of the deleterious effects of the SLC26a8 mutation.

TABLE 3
Transmission Ratio Distortion of Gnat3 Promoter and UTR Combination
with a SLC26a8 Dominant Negative Gene Mice Offspring

| Category | Observed | Expected |
|---|---|---|
| Negative | 16 | 8.5 |
| Positive | 1 | 8.5 |
| Total | 17 | 17 |

Transmission Ratio Distortion (TRD) = 94%

Proportion of motile sperm was assessed by independent fertility expert on sperm extracted from the epididymis of the transgenic mice with a SLC26a8 dominant negative gene. As shown in FIG. 4F, significant decreases in sperm motility were observed in transgenic mice with a SLC26a8dominant negative gene as compared to wild-type. Sperm cells of the transgenic mice with a SLC26a8dominant negative gene were also observed to have the characteristic structural defects at the midpiece.

Taken together, the results indicate that the Gnat3 -SLC26a8 dominant negative transgene in the parent mice successfully prevented transmission of the targeted chromosome to the offspring through RNA and protein tethering.

### Example 7: T-Complex Responder (TCR) Promoter 5′ and 3′ UTR Combination with a Slc26a8 Dominant Negative Gene

Because the TCR system is the best-studied transmission ration distortion model, TCR promoter and TCR 5′ and 3′ UTRs were used to test whether the tethering system is functional when introduced in a transgenic animal. The gene for TCR, Smok1, does not exist in non-rodents. Therefore, a Smok2b gene, from which the Smok1 gene is derived in wild-type mice and which has high sequence identity to a Smok1 gene is used.

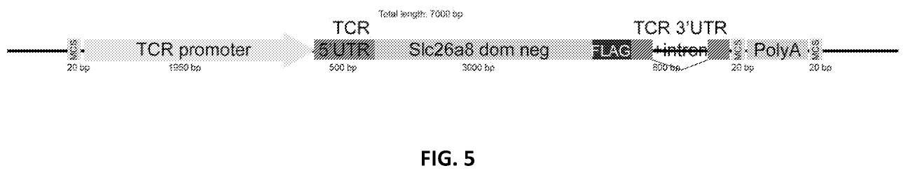

A construct was generated comprising the following Smok2b/TCR elements: the ˜2 kb promoter sequence upstream of the start codon, the ˜500 bp 5′UTR, and the ˜350 bp 3′UTR with the ˜500 bp intron naturally occurring in the 3′UTR. A Slc26a8 dominant negative gene was operably linked to the ˜2 kb Smok2b/TCR promoter sequence and a polyA site followed the 3′UTR (FIG. 5). Additionally, multiple cloning sites were introduced at each end of the construct and between the 3′UTR and the polyA site.

### Example 8: Odf1 Promoter and TCR 5′UTR and 3′ UTR Combination with a Slc26a8 Dominant Negative Gene

Because Odf1 is extremely strongly expressed very late in spermatogenesis and its promoter should preserve any timing effect required for the tethering to be effective. A construct similar to the construct of FIG. 5 but instead of the TCR promoter comprising the Odf1 promoter was generated. This construct allows the determination whether the tethering effect is in the UTR or the promoter.

### Example 9: Prevention and/or Inhibition of RNA Transfer by RNA Tethering to a Cytoskeletal Structure

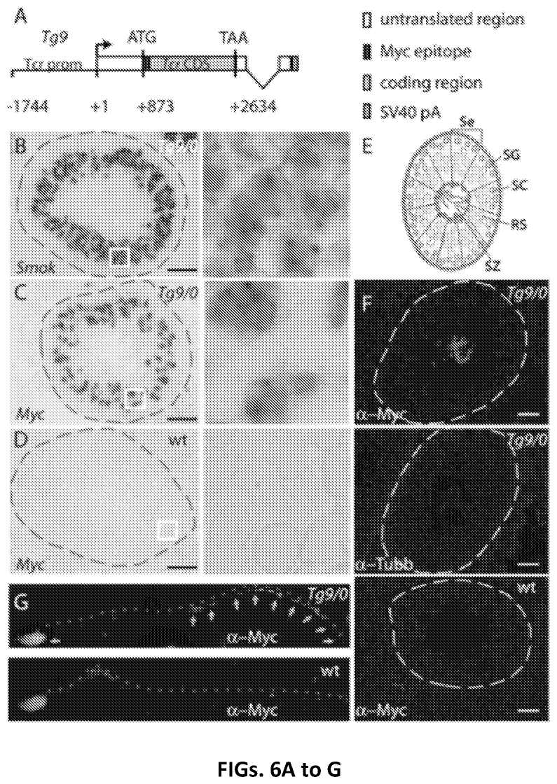

A genetic construct comprising elements of the Smok gene that encodes the t-Complex Responder (TCR) protein when introduced into a chromosome of a non-human transgenic animal prevents and/or inhibits transfer of a transcript to neighboring sperm cells through cytoplasmic bridges (3). The construct used comprised a TCR promoter, a specific 5′ UTR of 873 bp operably linked to a TCR gene and a myc tag. Histological cross sections of the seminiferous duct of wild-type and transgenic animals stained with antibodies against TCR and the myc tag demonstrated a ubiquitous presence of TCR in all sperm cells of the seminiferous tubules of wild-type animals but restriction of the presence of the myc tag to specific sperm cells present in the seminiferous tubules of transgenic animals (FIG. 6, reproduced from (3)).

### Example 10: Delay of Translation Until the Cytoplasmic Bridges Between Sperm Cells are No Longer Present

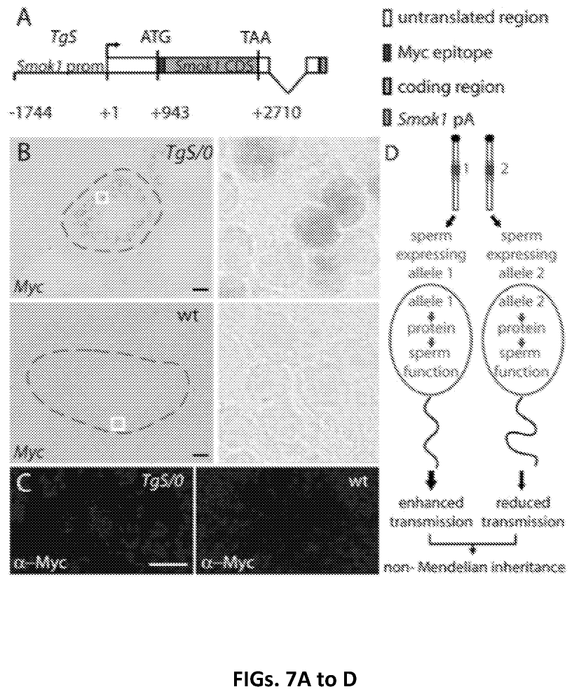

A genetic construct comprising elements of the Smok gene that encodes the t-Complex Responder (TCR) protein when introduced into a chromosome of a non-human transgenic animal demonstrated the restriction of the myc tag to specific sperm cells (3). The construct used comprised a TCR promoter, a specific 5′ UTR of 943 bp operably linked to a TCR gene and a myc tag. Histological cross sections of the seminiferous duct of wild-type and transgenic animals when stained with antibodies against the myc tag demonstrated the restriction of the presence of the myc tag to specific sperm cells present in the seminiferous tubules of transgenic animals and the absence of myc tag in wild-type animals (FIG. 7, reproduced from (3)).

A further genetic construct comprising instead of the TCR 5′UTR a Gnat3 5′UTR was constructed. Advantageously, the delay in translation occurred using both, the TCR 5′UTR and the Gnat3 5′UTR comprising constructs.

Statistical Analysis: values are shown as mean ±standard error of the mean as indicated. Differences between groups were analyzed using the nonparametric Mann-Whitney U test. Experiments were considered statistically significant if P values were <0.05. Calculations except 16s rDNA data were performed using Prism 5.0 software.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### Example 11: Goat Gnat3 Promoter and 5′UTR in Combination with a Goat SLC26a8 Dominant Negative Gene

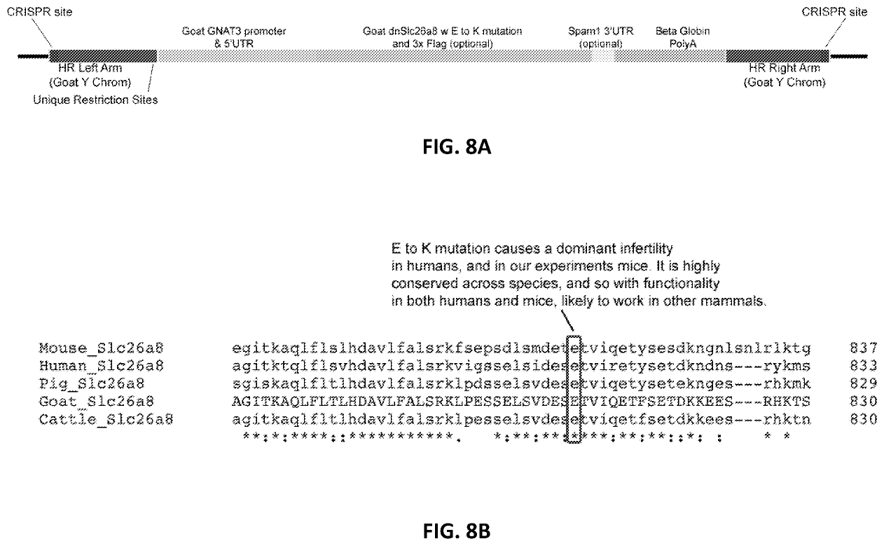

A genetic construct comprising elements of the goat Gnat3 promoter and 5′UTR in combination with a goat SLC26a8 dominant negative gene was created to prevent and/or inhibit transmission of an arbitrary chromosome in goat. As shown in FIG. 8A and SEQ ID NO. 1, the construct comprises sequentially: nucleotides 1-23 CRISPR site, 24-1079 left arm (match to goat Y chromosome), 1080-1087 NotI site, 1088-1121 FRT site, 1122-2811 goat Gnat3 promoter and 5′UTR (tethering region), 2812-5750 goat Slc26a8 with E to K mutation making dominant negative, 5751-5996 Spam1 3′UTR, 5997-7166 rabbit beta globin PolyA sequence (including last intron), 7167-7200 FRT site, 7201-7206 restriction site, 7207-8343 right arm (match to goat Y chromosome), and 8344-8366 CRISPR site. FIG. 8B shows by multiple sequence alignment that the E to K SLC26a8 amino acid mutation identified herewith is conserved among mouse, human, pig, goat, and cattle. Without wishing to be bound by any theory, it is postulated that the E to K mutation identified herewith renders SLC26a8 dominant negative in all placental mammals.

The insertion of the this construct into an arbitrary goat chromosome allows the prevention and/or inhibition of a fertilization event mediated by a goat sperm containing such chromosome and, thus prevents and/or inhibits the transmission of said chromosome to offspring in goat.

### Example 12: Alignment of the Gnat3 5′UTR Sequences from Mouse, Human, Cattle and Goat.

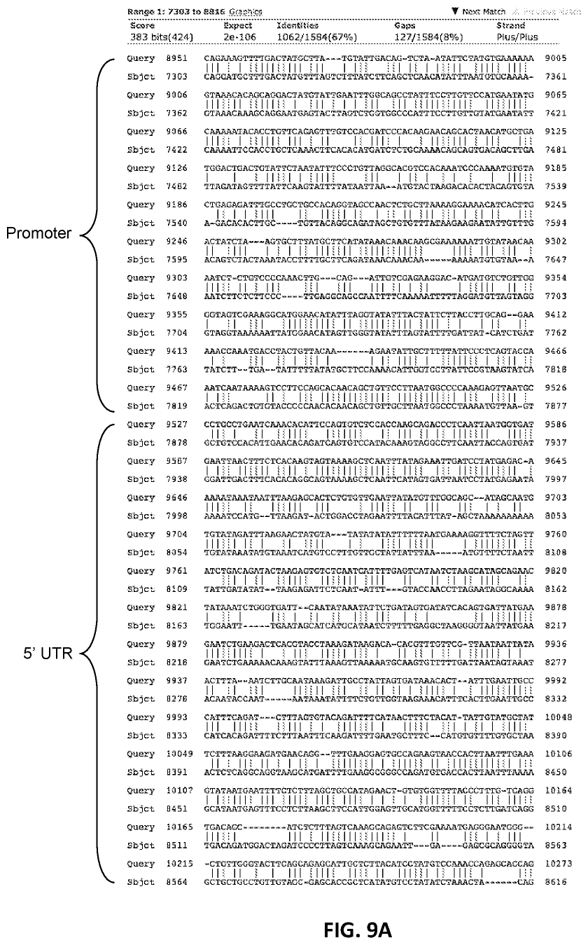

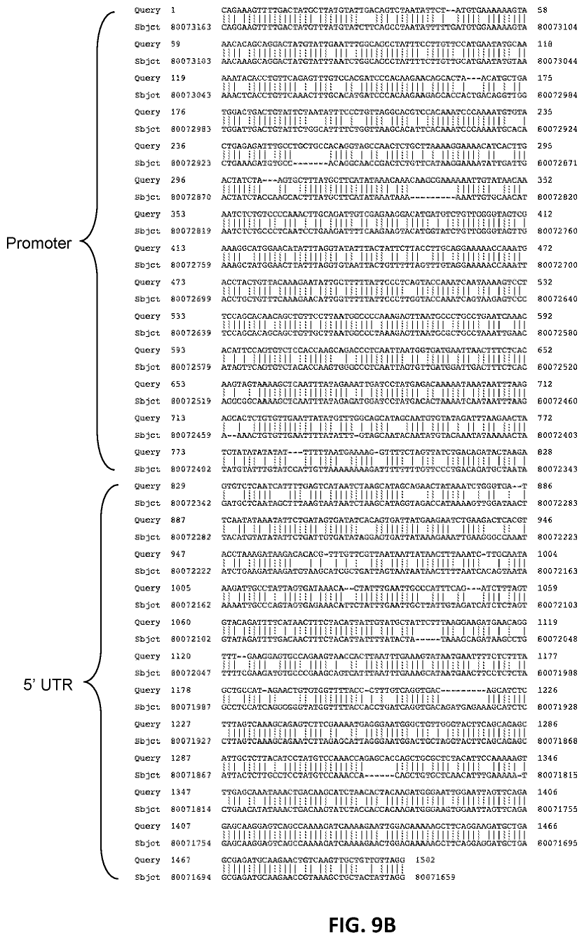

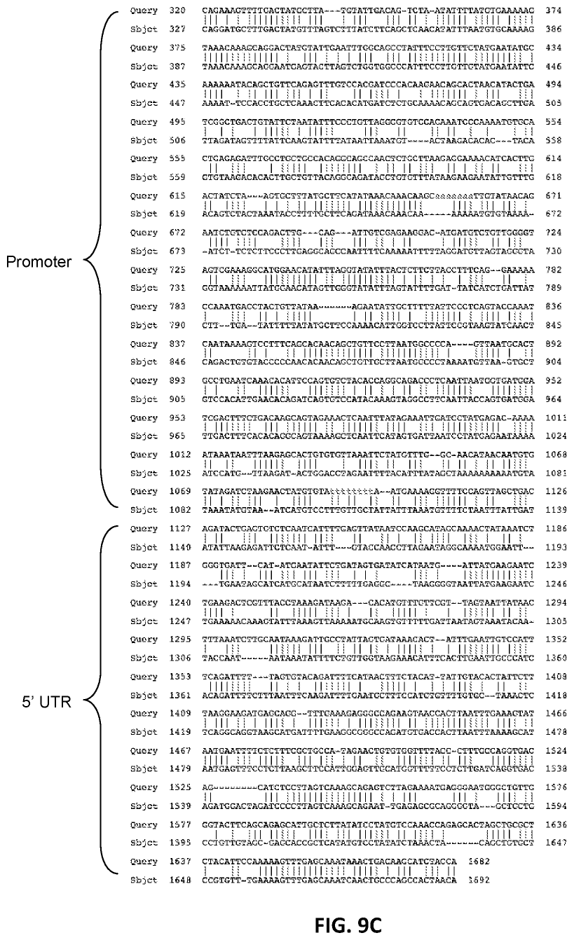

Sequence alignment of Gnat3 5′UTR sequences from goat (nucleotides 1122-2811 of SEQ ID NO. 1), mouse (SEQ ID NO. 2), human (SEQ ID NO. 3), and cattle (SEQ ID NO. 4) was performed. Pair-wise alignment is shown in FIGS. 9A (cattle-mouse alignment), 9B (cattle-human alignment), and 9C (goat-mice alignment). Surprisingly and remarkably, the Gnat3 5′UTR sequence showed a very high sequence homology between cattle, mouse, human, and goat, with as high as a 79% of identity in the cattle-human pairwise alignment (FIG. 9B). This finding is unexpected as non-coding regions are generally not well conserved across species. Without wishing to be bound by any theory, it is postulated that the Gnat3 5′UTR sequence is highly conserved in all placental mammals to which the method of the present invention applies.

### REFERENCES

1. Mosinger B, Redding K M, Parker M R, Yevshayeva V, Yee K K, Dyomina K, Li Y, Margolskee R F. Genetic loss or pharmacological blockade of testes-expressed taste gene causes male sterility. Proc Natl Acad Sci U S A. 2013 July 23;110(30):12319-24. doi: 10.1073/pnas.1302827110. Epub 2013 Jul. 1. PubMed PMID: 23818598; PubMed Central PMCID: PMC3725061.

2. Product information: anti-SLC26A8 antibody produced in rabbit. Sigma-Aldrich. Catalog number HPA038081.

3. Veron N, Bauer H, Weisse A Y, Luder G, Werber M, Herrmann B G. Retention of gene products in syncytial spermatids promotes non-Mendelian inheritance as revealed by the t complex responder. Genes Dev 2009; 23: 2705-10.

1-2. (canceled)

3. A method for preventing or forcing transmission of a particular chromosome in a male non-human transgenic mammal, comprising:
providing a genetic construct comprising an exogenous nucleic acid sequence operably linked to a promoter that activates expression of the exogenous nucleic acid sequence post-meiotically in a developing sperm cell;
wherein
(A) the exogenous nucleic acid sequence comprises an untranslated region (UTR) that tethers a transcript transcribed from the nucleic acid sequence to a cytoskeletal structure of the sperm cell; and the exogenous nucleic acid sequence encodes at least one protein that prevents transmission by inhibiting progressivity, motility, and/or penetration ability of a sperm cell or induces sperm cell death or forces transmission by promoting progressivity, motility, and/or penetration ability of a sperm cell or inhibits sperm cell death, and the at least one protein optionally comprises a membrane-association sequence that tethers to a cytoskeletal structure of the sperm cell; or
(b) wherein the exogenous nucleic acid sequence comprises (i) a short hairpin RNA (shRNA) or (ii) one small interfering RNA (siRNA) inserted into a micro RNA (miR) cassette, operably linked to an untranslated region (UTR) that tethers a transcript transcribed from the nucleic acid sequence to a cytoskeletal structure of the sperm cell, wherein the shRNA or the siRNA inserted into miR cassette prevents transmission by inhibiting expression of at least one gene involved in survival, motility, progressivity and/or penetration ability of the sperm cell or forces transmission by inhibiting at least endogenous one gene expressing a protein involved in survival, motility, progressivity and/or penetration ability of the sperm cell; and wherein the genetic construct further comprises a second exogenous nucleic acid sequence encoding the protein, and wherein for forcing transmission the second exogenous nucleic acid sequence encoding comprises third base wobbles such that the shRNA or siRNA inserted into miR cassette not inhibit expression of the second exogenous nucleic acid sequence encoding the protein;
and introducing the construct into the particular chromosome in a cell of the non-human animal.

4-6. (canceled)

7. The method of claim 3, wherein the particular chromosome is a sex chromosome or an autosome.

8.-9. (canceled)

10. The method of claim 3, wherein the genetic construct is introduced into the chromosome using a site-specific nuclease homologous recombination.

11. The method of claim 3, wherein the membrane-association sequence is a membrane-insertion sequence or a binding domain that binds to a protein or protein complex comprising a membrane-insertion sequence.

12. (canceled)

13. The method of claim 3, wherein the promoter is:
(a) a RNA polymerase III (pol III) promoter, a U6 promoter, or a H1 promoter;
(b) activates expression during late spermatogenesis, activates expression when cytoplasmic bridges between developing sperm cells are broken, or is selected from a promoter for Gnat3, Spergen-4, Spata19, the outer dense fiber of sperm tails 3b (Odf3b), the outer dense fiber of sperm tail 1(Odf1), the outer dense fiber of sperm tail 3 (Odf3), protamine, TNP-1, sperm mitochondria associated cysteine rich protein (smcp), and the testis specific promoter within the sixteenth intron of the cKIT gene; or
(c) a universal promoter that activates expression during late spermatogenesis or is selected from beta actin promoter, ubiquitin promoter, JeT promoter, SV40 promoter, beta globin promoter, elongation Factor 1 alpha (EF1-alpha) promoter, Mo-MLV-LTR promoter, Rosa26 promoter, and any combination of the foregoing.

14. (canceled)

15. The method of claim 3, wherein the exogenous nucleic acid sequence further comprises an untranslated region (UTR) that tethers a transcript transcribed from the nucleic acid sequence to a cytoskeletal structure of the sperm cell.

16-21. (canceled)

22. The method of claim 3, wherein the UTR:
(a) is linked to the 5′ side or the 3′ side of the nucleic acid sequence that encodes the at least one protein;
(b) delays translation of the at least one protein until the cytoplasmic bridges between developing sperm cells are broken; and/or
(c) is selected from a t-Complex Responder, Gnat3, Tas1r3, and Spam 1 gene.

23-26. (canceled)

27. The method of claim 3, wherein the at least one protein is:
(a) a dominant-negative form of a protein that enables and/or promotes survival, progressivity, motility, or penetration ability of a sperm cell;
(b) a dominant-negative protein selected from the group consisting of dominant negative SUNS, a dominant negative mutant Sept4, a dominant negative Sept12, a dominant negative CATSPER1, a dominant negative CATSPER2, a dominant negative SLC26A8, a dominant negative Spata16, a dominant negative PLCZ1, a dominant negative DPY19L2, and/or a dominant negative form of Gpx4, a dominant negative form of Hook1, a dominant negative form of Prrs21, a dominant negative form of Oaz3, a dominant negative form of Cntrob, and a dominant negative form of Ift88; or
(c) is a dominant-negative Slc26a8 protein.

28-29. (canceled)

30. The method of claim 3, wherein the cell is a spermatogonial stem cell, the male non-human transgenic mammal is a sterile, hybrid male recipient animal, and the introducing step comprises:
providing the spermatgonial stem cell from a male donor animal;
introducing the genetic construct into the spermatogonial stem cell, wherein the nucleic acid construct is introduced into the particular chromosome;
introducing the donor spermatogonial stem cell into a reproductive organ of the sterile, hybrid male recipient animal, wherein donor spermatogonial stem cell produces donor-derived, fertilization-competent, haploid sperm cells lacking the particular chromosome from the sterile, hybrid male recipient animal, wherein the hybrid animal has at least one parentage that is from the same genus as the donor animal;
optionally, collecting the donor-derived, fertilization-competent, haploid sperm cells produced by the sterile, hybrid male recipient animal; and
optionally, fertilizing an egg using the collected donor-derived, fertilization-competent, haploid sperm cells.

31. A genetic construct comprising a nucleic acid sequence operably linked to a promoter that activates expression of the nucleic acid sequence post-meiotically in a developing sperm cell, wherein the nucleic acid sequence comprising an untranslated region (UTR) that tethers a transcript transcribed from the nucleic acid sequence to a cytoskeletal structure of a sperm cell; and
wherein the nucleic acid sequence encodes a dominant-negative Slc26a8 protein or at least one protein that inhibits progressivity, motility, and/or penetration ability of a sperm cell or induces sperm cell death or encodes at least one protein that promotes progressivity, motility, and/or penetration ability of a sperm cell or inhibits sperm cell death, and the at least one protein optionally comprises a membrane-insertion sequence that tethers to a cytoskeletal structure of the sperm cell.

32. (canceled)

33. The genetic construct of claim 31, wherein the promoter is selected from a promoter for Gnat3, Spergen-4, Spata19, the outer dense fiber of sperm tails 3b (Odf3b), the outer dense fiber of sperm tail 1(Odf1), the outer dense fiber of sperm tail 3 (Odf3), protamine, TNP-1, sperm mitochondria associated cysteine rich protein (smcp), t-Complex responder and the testis specific promoter within the sixteenth intron of the cKIT gene.

34. (canceled)

35. The genetic construct of claim 31, wherein the UTR is linked to the 5′ side of the nucleic acid sequence that encodes the at least one protein and/or is selected from a t-Complex Responder, Gnat3, Tas1r3, and Spam1 gene.

36-39. (canceled)

40. A genetic construct comprising a nucleic acid sequence operably linked to a RNA polymerase III (pol III) promoter, wherein the nucleic acid sequence optionally comprising an untranslated region (UTR) that tethers a transcript transcribed from the nucleic acid sequence to a cytoskeletal structure of a sperm cell; and
wherein the nucleic acid sequence comprises a short hairpin RNA (shRNA) promoting survival, motility, progressivity and/or penetration ability of the sperm cell.

41. The genetic construct of claim 40, wherein the pol III promoter is selected from a U6 promoter and a H1 promoter.

42. The genetic construct of claim 40, wherein the at least one protein is selected from Tas1R3 and Gnat3.

43. The genetic construct of claim 40, comprising at least two shRNAs and wherein the at least two shRNAs are for Tas1R3 and Gnat3, or wherein the shRNA is an shRNA for Gnat3; the nucleic acid further comprising a nucleic acid sequence encoding a Gnat3 protein operably linked to a Gnat3 promoter, wherein the nucleic acid sequence encoding the Gnat3 protein comprises third base wobbles such that the shRNA for Gnat3 does not bind said nucleic acid sequence encoding the Gnat3 protein.

44. (canceled)

45. The genetic construct of claim 31, wherein the genetic construct is targeted to a deleterious gene or allele for prevention of transmission or to a favorable gene or allele for forced transmission.

46. (canceled)

47. The genetic construct of claim 31, wherein the genetic construct is inserted in a sex chromosome or an autosome.

48. A nucleic acid molecule comprising at least one small interfering RNA (siRNA) for at least one protein that enables progressivity, motility and/or penetration ability of a sperm cell; wherein the at least one siRNA is inserted into a micro RNA (miR) cassette, which miR cassette comprises at least one sequence homologous to a sequence of a 3′UTR region of a gene expressed in late spermatogenesis.

49. A method of producing fertilization-competent haploid sperm cells, comprising:
providing spermatgonial stem cell from a male donor animal;
providing a genetic construct of claim 31;
introducing the genetic construct into a spermatogonial stem cell obtained from the male donor animal, wherein the genetic construct is introduced into a sex chromosome;
providing a sterile, hybrid male recipient animal, wherein the hybrid animal has at least one parentage that is from the same genus as the donor animal;
introducing the donor spermatogonial stem cell into a reproductive organ of the sterile, hybrid male recipient animal that produces donor-derived, fertilization-competent, haploid sperm cells; and
collecting the donor-derived, fertilization-competent, haploid sperm cells produced by the sterile, hybrid male recipient animal.

50. The method of claim 3, wherein the genetic construct is targeted to a deleterious gene or allele in the particular chromosome for prevention of transmission of the particular chromosome or to a desired gene or allele in the particular chromosome for forced transmission.

51. (canceled)

52. The method of claim 7, wherein the genetic construct is targeted to a site specific to the Y chromosome or to the X chromosome.

53. (canceled)
